SEQUENCE LISTING (12 sequences)

SEQ ID NO: 1 - DNA, 105 bp, Artificial Sequence
  Template DNA originating from M13mp18
acaggaaacagctatgaccatgattacgaattcgagctcggtacccggggatcctctagagtcgacctgcaggcatgcaagcttggcactggccgtcgttttaca

SEQ ID NO: 2 - DNA, 17 bp, Artificial Sequence
  DNA primer complementary with base sequence between 88 and 105 of SEQ ID NO1, but the base replaced C at 15 of this DNA primer by G for introducing a mismatch between DNA primer and template
tgtaaaacgacggcgag

SEQ ID NO: 3 - DNA, 17 bp, Artificial Sequence
  DNA primer complementary with base sequence between 88 and 105 of SEQ ID NO1, but the base replaced C at 15 of this DNA primer by G for introducing a mismatch between DNA primer and template
tgtaaaacgacggcgat

SEQ ID NO: 4 - DNA, 17 bp, Artificial Sequence
  DNA primer complementary with base sequence between 88 and 105 of SEQ ID NO1, and DNA primer being able to be extended
tgtaaaacgacggccag

SEQ ID NO: 5 - DNA, 95 bp, Artificial Sequence
  Template DNA originating from p53 and including base sequence of exon 8
ctttcttgcggagattctcttcctctgtgcgccggtctctcccaggacaggcacaaacacgcacctcaaagctgttccgtcccagtagattacca

SEQ ID NO: 6 - DNA, 22 bp, Artificial Sequence
  DNA primer complementary with base sequence between 55 and 76 of SEQ ID NO5, but the base replaced T at 19 of this DNA primer by A for introducing a mismatch between DNA primer and template
aacagctttgaggtgcgtgatt

SEQ ID NO: 7 - DNA, 95 bp, Artificial Sequence
  Template DNA originating from p53 and including base sequence of exon 8, but the base replaced A at 55 of this template DNA by T
ctttcttgcggagattctcttcctctgtgcgccggtctctcccaggacaggcactaacacgcacctcaaagctgttccgtcccagtagattacca

SEQ ID NO: 8 - DNA, 22 bp, Artificial Sequence
  DNA primer complementary with base sequence between 55 and 76 of SEQ ID NO7, but the base replaced T at 19 of this DNA primer by A for introducing a mismatch between DNA primer and template
aacagctttgaggtgcgtgata

SEQ ID NO: 9 - DNA, 22 bp, Artificial Sequence
  DNA primer complementary with base sequence between 55 and 76 of SEQ ID NO5 and 7, but the base replaced T at 19 of this DNA primer by A for introducing a mismatch between DNA primer and
aacagctttgaggtgcgtgatc

SEQ ID NO: 10 - DNA, 22 bp, Artificial Sequence
  DNA primer complementary with base sequence between 55 and 76 of SEQ ID NO5 and 7, but the base replaced T at 19 of this DNA primer by A for introducing a mismatch between DNA primer and
aacagctttgaggtgcgtgatg

SEQ ID NO: 11 - DNA, 63 bp, Artificial Sequence
  Template DNA originating from p53 and including base sequence of exon 8
gtggtaatctactgggacggaacagctttgaggtgcgtgtttgtgcctgtcctgggagagacc

SEQ ID NO: 12 - DNA, 20 bp, Artificial Sequence
  DNA primer complementary with base sequence between 44 and 63 of SEQ ID NO11, and DNA primer being able to be extended
ggtctctcccaggacaggca

(19) United States
(12) Patent Application Publication
(10) Pub. No.: US 2003/0165861 A1
(43) Pub. Date: Sep. 4, 2003

(54) Method of analysis of DNA sequence

(75) Inventors: Yuki Wakabayashi, Inagi (JP); Hideki Kambara, Hachioji (JP); Guohua Zhou, Kokubunji (JP); Masao Kamahori, Kokubunji (JP)

Correspondence Address: Stanley P. Fisher, Reed Smith Hazel & Thomas LLP, Suite 1400, 3110 Fairview Park Drive, Falls Church, VA, 22042-4503, US

(73) Assignee: Hitachi, Ltd.

(21) Appl. No.: 10/038,717

(22) Filed: Jan. 8, 2002

(30) Foreign Application Priority Data
Apr. 16, 2001 (JP) 2001-117232

(51) Int. Cl. C12Q 1/68; C12P 19/34; C12N 9/04

(52) U.S. Cl. 435/6; 435/91.2; 435/190

(57) ABSTRACT

For provision of a method of analysis of DNA sequence, reagent kit used therefore and DNA sequence analyzer for detecting single nucleotide polymorphysms (SNPs) of DNA, reagents for complementary strand extension reaction and chemiluminescence-reaction are pretreated with pyrophosphatase and apyrase. An impurity PPi which disturbs the chemiluminescence-reaction is degraded by pyrophosphatase, while an impurity ATP is degraded by apyrase. The reagents thus pretreated are used for the complementary strand extension reaction and chemiluminescence-reaction.

### BACKGROUND OF THE INVENTION

The present invention relates to a method of analysis of DNA sequence, a reagent kit used therefore and an analyzer for carrying out the method. In particular, the invention pertains to a method of analysis of DNA sequence by detecting pyrophosphoric acid (PPi), which has been generated upon formation of an extended strand of a primer hybridized to DNA through a complementary strand, by making use of chemiluminescence-reaction; a reagent kit used therefore; and an analyzer for carrying out the method. More specifically, the invention pertains to a method of determination of a DNA sequence, a method of detecting single nucleotide polymorphisms (SNPs), a method of measuring gene expression frequency of single nucleotide polymorphisms, and an analyzer for carrying out this analyzing method.

With a recent progress in the genome analysis, there is an active movement of widespread using of DNA data for medical treatment. In particular, detection of a specific sequence in genome or detection of SNPs in a specific sequence has been an important theme. Based on the presumption that SNPs in human genome occur once per 1000 bases, 3 million to 10 million SNPs are considered to exist in human genome. These SNPs have a relation with individual variation or sensitivity to pharmaceuticals. Recently, analysis of SNPs has therefore attracted attentions.

Analysis of SNPs include, for example, analysis (1) for detecting SNPs and their frequent expressing site in a base sequence, analysis (2) for finding important SNPs data in medical fields among the SNPs data base obtained, and analysis (3) for obtaining a guideline for diagnosis or treatment of each patient by using the important SNPs in medical fields.

In each country, searching for SNPs in analysis (1) is now carried out using DNA sequencing or SSCP (single strand conformation polymorphism analysis). With regards to analysis (3), discussion on a systematic gene mapping method based on analysis data of SNPs has started for identification of a disease gene. Development of an SNPs typing method (a method to study the existence of mutation at a site on which mutation is presumed to exist) with higher throughput and higher accuracy is considered to be inevitable and various analyzing methods have already been proposed.

Examples include (1) SNP-ARMS (Amplification refractory mutation system) method of hydridizing a genotyping primer which is hybridized specifically to a particular base sequence to a target DNA, thereby conducting a complementary strand extension reaction and analyzing, by gel electrophoresis, the product of the complementary strand extension reaction whether it has mutation or not; (2) MALDI-TOF/MS method (Matrix Assisted Laser Desorption Inonization-Time of Flight/Mass Spectroscopy) of hydridizing a primer to target DNA, thereby carrying out complementary strand extension reaction of only one base and analyzing, by mass spectroscopy, the product of the complementary strand extension reaction, (3) In TaqMan PCR method DNA probe is cleaved by an enzyme at a mutation expected site and the degraded product is fluorescently detected to attain high sensitivity analysis, (4) Invader method of using two non-fluorescent labeling oligomers in the Taqman PCR method, (5) RCA (Rolling circle amplification) method of detecting SNPs, by the amplification reaction, based on the presence or absence of formation of a cyclic single stranded DNA, and (6) pyrosequencing method (Anal. Biochemistry, 280, 103-110(2000)) of hybridizing a primer to target DNA, converting pyrophosphoric acid, which has been generated by a complementary strand extension reaction, to adenosine 5′-triphosphate, reacting the adenosine 5′-triphosphate with a luminescent reagent such as luciferin, measuring the intensity of luminescence emitted by the reaction, and determining the base sequence successively from the site adjacent to the primer. The above-exemplified methods are described in detail in “GENOME SCIENCE ON POST SEQUENCING (1) STRATEGY OF SNPs: GENE polymorphisms” (ed by NAKAMURA Yusuke, pp. 93-149 (2000), published by Nakayama Shoten, Tokyo).

Japanese Patent Laid-Open No. 203998/1995 includes a description on observation, by luminescence-reaction, of n pieces of pyrophosphoric acid liberated by the extension of n pieces of bases as a result of hybridization of bases of a nucleic acid specimen with bases complementary thereto and extension reaction of a primer in one direction. Japanese Patent Laid-Open No. 000299/1996 includes a description on enzymatic decomposition and removal in advance of pyrophosphoric acid, which exists in a sample, by using pyrophosphatase or the like.

### SUMMARY OF THE INVENTION

Analysis (2) requires study of SNPs at a vast number of sites concerning a vast number of Template DNAs. For example, it is necessary to carry out, in a vast number of times, SNPs analysis of many patients belonging to a certain disease group and many normal volunteers as a control and study whether the target SNPs have a medical significance or not based on the above results. Analysis (2) therefore needs development of a high throughput and low running-cost system.

It is also necessary to study a probability of mutation by detecting SNPs from the mixture of a number of Template DNAs classified by the characteristics of patients and find a correlation between this probability and a disease or sensitivity to pharmaceuticals. This requires a highly accurate method for quantitative analysis, but such a method does not exist at present.

Among the related art, a pyrosequencing method leads to an apparatus of a low running cost and high throughput. Since this pyrosequencing method does not require a high voltage power supply, laser light source, fluorescent reagent and expensive mass spectroscopy, it facilitates size and cost reduction of the apparatus. The pyrosequencing method is however accompanied with such a problem as a detection limit due to impurities, pyrophosphoric acid (which will hereinafter be abbreviated as “PPi”) generated by an undesirable chemical change due to the impurities, and PPi and adenosine 5′-triphosphate (which will hereinafter be abbreviated as “ATP”) contained, as impurities, in a reagent kit. Above all, PPi contained in deoxynucleotides (nucleic acid substrates, dNTPs: N=A, C, G, T) serving as a substrate of a complementary strand extension reaction using a DNA polymerase imposes a limitation on detection of PPi produced by extension reaction of nucleic acid substrates.

An object of the present invention is to provide a method of analysis of DNA sequence which needs only a low running cost, has excellent determination accuracy, and is capable of detecting SNPs with high sensitivity and high throughput owing to an improvement in detection limit; a reagent kit using the method; and an analyzer for carrying out this analysis method.

In one aspect of the present invention, there are thus provided a method of analysis of DNA sequence, a reagent kit and an analyzer for executing this method, each to be applied to a pyrosequencing method. In another aspect of the invention, there are also provided a method of analysis of DNA sequence, a reagent kit and an analyzer for executing this method, each to be applied to SNPs expression frequency analyzing method (BAMPER method: Bioluminometric Assay coupled with Modified Primer Extension Reaction) (Japanese Patent Application No. 300577/2000).

In this BAMPER method, used are artificial mismatched primers whose second or third base from the 3′ terminus thereof are artificially substituted by a base not complementary to the base of a target nucleic acid (single stranded DNA template). The artificially substituted base imparts the artificial mismatched primers, which have been hybridized to the target nucleic acid via complementary strands, with switching characteristics between promotion and termination of extension reaction of the artificial mismatched primers.

The BAMPER method can attain a high detection sensitivity, because in this method, luminescence due to a large amount of PPi formed by continuous complementary strand extension of several hundreds of bases is detected by chemiluminescence-reaction. It can therefore be applied to a trace amount of single stranded DNA (ssDNA) templates and detection of mutation. Similar to the pyrosequencing method, the detection limit of this method is determined by PPi or ATP contained as impurities in reagents used in this method.

In the method of analysis of DNA sequence according to the present invention, PPi contained in a reagent used for extension reaction of a DNA primer and a reagent used for the pyrosequencing method is degraded in advance by using pyrophosphatase (which will hereinafter be abbreviated as “PPase”) and/or ATP contained in the above-described reagents is degraded in advance by using apyrase. The method of analysis of DNA sequence according to the present invention can be applied to analysis of the base sequence of nucleic acid, analysis of polymorphism of nucleic acid bases and analysis of expression frequency of polymorphism of nucleic acid bases.

In the reagent kit according to the present invention, PPase and/or apyrase contained therein degrades PPi or ATP, which has been contained in the reagent as impurities, with a good efficiency so that highly sensitive detection and improvement of determination accuracy can be realized. In addition, a low running cost and high thorough-put can be attained.

For SNPs analysis, study of existence or absence of gene mutation is necessary. In the present invention, PPi generated by the extension reaction of a primer—which is hybridized specifically to the SNPs site of DNA and is capable of controlling the complementary strand extension reaction depending on the presence or absence of mutation—is detected by chemiluminescence-reaction using luciferase. When the template DNA has the target mutation, chemiluminescence due to continuous formation of extended strands is detectable.

When SNPs are analyzed using primers specific to mutation-free wild type DNA and mutant DNA, amounts of them can be detected respectively. It is therefore possible to find a ratio of mutant DNA and wild type DNA even if a mixed sample is used.

Application of the present invention to the BAMPER method attains sensitivity of at least 2 figures greater than the pyrosequencing method, because extension of primers is carried out continuously by complementary strand extension reaction and by using a large number of PPi thus generated, chemiluminescence-reaction is effected.

The preferred embodiments of the invention will next be enumerated.

(1) In the method of analysis of DNA sequence according to the present invention, PPi contained in a reagent used for extension reaction of a DNA primer, which has been hybridized to a target nucleic acid (DNA or RNA) through a complementary strand, is degraded in advance by using PPase and/or ATP contained in a reagent is degraded in advance by using apyrase. After this pretreatment, PPase and/or apyrase are removed. PPase and/or apyrase have been immobilized to a solid, e.g. beads, magnetic beads or the like, which facilitates addition or removal of PPase and/or apyrase from each reagent solution. After completion of the pretreatment and removal of PPase and/or apyrase, extension reaction is effected and chemiluminescense by PPi generated by this reaction is detected.

(2) In the method of analysis of DNA sequence according to the present invention, (a) PPase is added to each of one or more solutions containing different deoxynucleotides (dNTP, N=A, C, G, T), respectively, or each of one or more solutions containing different deoxynucleotides, respectively, at least one of which is an analogue thereof, to degrade PPi contained in the solution; and/or (b) apyrase is added to degrade ATP contained in each of the solutions. As in (1), PPase and/or apyrase is removed after the pretreatment.

By using a DNA primer, DNA polymerase and at least one pretreated solution, the DNA primer which is hybridized to a target nucleic acid (DNA or RNA) via a complementary strand is extended by extension reaction. In the presence of adenosine 5′-phosphosulfate (which will hereinafter be abbreviated as “APS”) and ATP sulfurylase, the PPi generated by the extension reaction is converted to ATP and, then, emits light by the chemiluminescence-reaction including ATP, luminescent enzyme (such as luciferase) and luminescent substrate (such as luciferin). This luminescence is detected.

In the above-described example, the 3′ terminus base of the primer is complementary to the base which exists one base behind the 3′ terminus side of the single nucleotide polymorphism site of the target nucleic acid. The second or third base from the 3′ terminus of the DNA primer has been substituted with a base not complementary to the base sequence of the target nucleic acid.

(3) In the method of analysis of DNA sequence according to the present invention, (a) PPase is added to each of a solution containing deoxyadenosine 5′-α-triphosphate (which will hereinafter be abbreviated as “dATPαS”), a solution containing deoxythymidine 5′-triphosphate (which will hereinafter be abbreviated as “dTTP”), a solution containing deoxyquanosine 5′-triphosphate (which will hereinafter be abbreviated as “dGTP”), and a solution containing deoxycytidine 5′-triphosphate (which will hereinafter be abbreviated as “dCTP”) to degrade PPi contained in each of the solutions as a pretreatment; and/or (b) apyrase is added to degrade ATP contained in each of the solutions as a pretreatment. After the pretreatment, PPase and/or apyrase in each of the solutions is removed, similar to (1).

By using a DNA primer, DNA polymerase and at least one pretreated solution, the DNA primer which has been hybridized to the target nucleic acid (DNA or RNA) via a complementary strand is extended by extension reaction. In the presence of APS and ATP sulfurylase, the PPi generated by the extension reaction is converted to ATP and emits light by the chemiluminescence-reaction including ATP, luciferase and luciferin. This luminescence is detected.

(4) In the method of analysis of DNA sequence according to the present invention, (a) PPase is added to a solution containing dATPαS, dTTP, dGTP, and dCTP to degrade PPi contained in the solution as a pretreatment; and/or (b) apyrase is added to degrade ATP contained in the solution as a pretreatment. After the pretreatment, PPase and/or apyrase in the solution is removed similar to (1).

By using a DNA primer, DNA polymerase and the pretreated solution, the DNA primer which has been hybridized to a target nucleic acid (DNA or RNA) via a complementary strand is extended by extension reaction. In the presence of APS and ATP sulfurylase, the PPi generated by the extension reaction is converted to ATP and luminescence caused by the chemiluminescence-reaction including ATP, luciferase and luciferin is detected.

In the above-described example, the second or third base from the 3′ terminus of the DNA primer has been substituted by a base not complementary to the base sequence of the target nucleic acid. The strand extended by the extension reaction is degraded from the 5′ terminus by the 5′ → 3′ exonuclease reaction and complementary strand hybridization of the DNA primer to the target nucleic acid is repeated to effect extension reaction. In this manner, the light emitted by the chemiluminescence-reaction is detected.

(5) In the method of analysis of DNA sequence according to the present invention, pretreatment is conducted (a) by adding PPase to a solution containing deoxyadenosine 5′-triphosphate (which will hereinafter be abbreviated as “dATP”), dTTP, dGTP and dCTP to degrade PPi contained in the solution; and/or (b) by adding apyrase to the solution to degrade ATP contained therein. After the pretreatment, PPase and/or apyrase in the solution is removed as in (1).

By using a DNA primer, DNA polymerase and the pretreated solution, the DNA primer which has been hybridized to the target nucleic acid (DNA or RNA) via a complementary strand is extended by extension reaction. Chemiluminescence due to the PPi generated by the extension reaction is detected.

In the above-described case, instead of any one of dATP, dTTP, dGTP and dCTP, an analogue thereof is usable.

In the above-described example, the second or third base from the 3′ terminus of the DNA primer has been substituted by a base not complementary to the base sequence of the target nucleic acid. The strand extended by the extension reaction is degraded at the 5′ terminus by the 5′ → 3′ exonuclease reaction and complementary strand hybridization of the DNA primer to the target nucleic acid is repeated to effect extension reaction. In this manner, the light emitted by the chemiluminescence-reaction can be detected.

(6) In the method of analysis of DNA sequence according to the present invention, pretreatment is conducted (a) by adding PPase to a solution containing dATP, dTTP, dGTP and dCTP to degrade PPi contained in the solution; and/or (b) by adding apyrase to the solution to degrade ATP contained therein. After the pretreatment, PPase and/or apyrase in the solution is removed as in (1).

In the above-described example, a first oligomer having a complementary strand extending capacity and falling within a range of five bases to 8 bases and a second oligomer forming complementary strand hybridization to a target nucleic acid (DNA or RNA) and not having a complementary strand extending capacity are hybridized in series to the target nucleic acid through a complementary strand. By using DNA polymerase and the pretreated solution, extension reaction of the first oligomer is conducted. The PPi generated by this extension reaction is detected by chemiluminescence-reaction.

In the above-described example, any one of dATP, dTTP, dGTP and dCTP can be substituted by an analogue thereof. The second or third base from the 3′ terminus of the first oligomer has been replaced with a base not complementary to the base sequence of a predetermined site of the target nucleic acid.

In any one of the above-described embodiments (1) to (6), removal of PPase and/or apyrase is conducted after the pretreatment. Alternatively, an inactivator of PPase and/or apyrase may be added. As shown later in specific examples, remaining of a small amount of PPase and/or apyrase in the solution doe not cause any problem so that the removal treatment of PPase and/or apyrase after pretreatment may be omitted, though depending on its (or their) amount.

(7) The reagent kit according to the present invention has PPase in one or more solutions containing different deoxynucleotides (dNTP, N=A, C, G, T), respectively, or in one or more solutions containing different deoxynucleotides, respectively, at least one of which is an analogue thereof.

(8) The reagent kit according to the present invention has PPase in one or more solutions containing different deoxynucleotides (dNTP, N=A, C, G, T), respectively, or in one or more solutions containing different deoxynucleotides, respectively, at least one of which is an analogue thereof. A solution containing DNA polymerase may be added to the reagent kit.

(9) The reagent kit according to the present invention has dATPαS, dTTP, dGTP, dCTP and PPase. The PPase has been immobilized on a solid.

(10) The reagent kit according to the present invention has dATP, dTTP, dGTP, dCTP and PPase and/or apyrase. Instead of any one of dATP, dTTP, dGTP and dCTP, its analogue is usable.

The reagent kits of the present invention as described in (7) to (10) are each used for a method of analysis of DNA sequence wherein PPi generated by extension reaction of a DNA primer hybridized to a DNA primer through a complementary strand is detected by the chemiluminescence-reaction.

(11) The reagent kit of the present invention contains at least one of DNA polymerase, DNA primer, APS, ATP sulfurylase, luciferase, luciferin and apyrase and PPase and is used for the method of analysis of DNA sequence wherein PPi generated by the extension reaction of the DNA primer, which has been hybridized to a target nucleic acid through a complementary strand, is converted to ATP and a light generated by chemiluminescence-reaction including ATP, luciferase and luciferin is detected. PPase and/or apyrase has been immobilized on a solid.

### BRIEF DESCRIPTION OF THE DRAWINGS

FIG. 1 is a flow chart for describing a process for preparing a noise-reduced template solution in the method of analysis of DNA sequence by detecting PPi, which has been generated by complementary strand extension, by chemiluminescence-reaction, in Embodiment 1 of the present invention;

FIGS. 2A to 2C each illustrates a principle of detecting PPi, which has been generated by single nucleotide extension of a primer, by chemiluminescence-reaction, in Embodiment of the present invention;

FIGS. 3A, 3B and 3C each illustrates examples of the method and apparatus of pretreatment, in Embodiment 1 of the present invention, wherein degradation of impurities contained in a reagent or Template DNA is conducted with pyrophosphatase and apyrase by using a solid phase or membrane;

FIG. 4 illustrates comparison of noise signals between presence and absence of pretreatment with PPase in Embodiment 1 of the present invention;

FIG. 5 illustrates dependence of noise signals on the concentration of dCTP after pretreatment with PPase in Embodiment 1 of the present invention;

FIG. 6 is a schematic view of the constitution of a DNA sequencer employed for pyrosequencing, in Embodiment 1 of the present invention;

FIG. 7 illustrates detection of a strand extended by pyrosequencing, in Embodiment 1 of the present invention;

FIGS. 8A and 8B are each a schematic view for describing a principle of a measuring method of SNPs expression frequency by the primer extension method, in Embodiment 2 of the present invention;

FIGS. 9A and 9B each illustrates a principle of a high-sensitivity measuring method of SNPs expression frequency by the primer extension method using an artificial mismatched primer, in Embodiment 2 of the present invention;

FIG. 10 illustrates a principle of a high sensitivity measuring method of SNPs expression frequency by the primer extension method using an artificial mismatched primer and 5′ → 3′exonuclease, in Embodiment 2 of the present invention;

FIG. 11 illustrates effects of the pretreatment with PPase in SNPs detection by the primer extension method using an artificial mismatched primer, in Embodiment 2 of the present invention;

FIG. 12 illustrates comparison of sensitivity of SNPs detection between the primer extension method using an artificial mismatched primer and pyrosequencing, in Embodiment 2 of the present invention;

FIGS. 13A and 13B illustrate comparison between use of a conventional primer and use of an artificial mismatched primer for determining real luminescence intensity and pseudo-luminescence (false-positive-luminescence) intensity by the measurement of SNPs expression frequency, in Embodiment 2 of the present invention;

FIG. 14 is a graph illustrating analysis results of SNPs expression frequency in accordance with the BAMPER method by using an artificial mismatched primer, in Embodiment 2 of the present invention;

FIG. 15 illustrates a method of measuring, in one reaction vessel, a plurality of SNPs existing in one DNA or a plurality of DNAs, in Embodiment 3 of the present invention;

FIG. 16 illustrates comparison of signal intensity among the varied amounts of PPase added in the pyrosequencing method and also that between presence and absence of PPase removing operation, in Embodiment 1 of the present invention; and

FIGS. 17A to 17D each illustrates an automated DNA sequencer including a step of treating a reaction reagent by using PPase and apyrase in the present invention, wherein FIG. 17A illustrates the whole constitution and FIGS. 17B to 17D each illustrates the removing part of an impurity such as PPi or ATP from the reagent.

### DESCRIPTION OF THE PREFERRED EMBODIMENT

Examples of the present invention will next be described more specifically with reference to accompanying drawings.

Embodiment I

In Embodiment I, a primer hybridized to template DNA (target DNA) is extended and a large amount of PPi generated by extension is detected by chemiluminescence-reaction. Although use of chemiluminescence-reaction essentially accomplishes high sensitivity detection, various impurities contained in a reagent become a luminescence source and the detection results tend to contain a noise. In high sensitivity measurement, these impurities must be degraded and removed from the reagent. High sensitivity measurement enables analysis only with trace amounts of a reagent and template, thereby actualizing a low-cost detection system. As a result of various investigations, it has been proved that unnecessary luminescence leading to a noise is caused by PPi contained in a reagent dNTP (N=A, C, G, T) or ATP contained in an enzyme such as polymerase. It is therefore important to carry out chemiluminescence-reaction after degradation of impurities, ATP and PPI, contained in a reagent and removal of them from the reagent and then, to measure the luminescence.

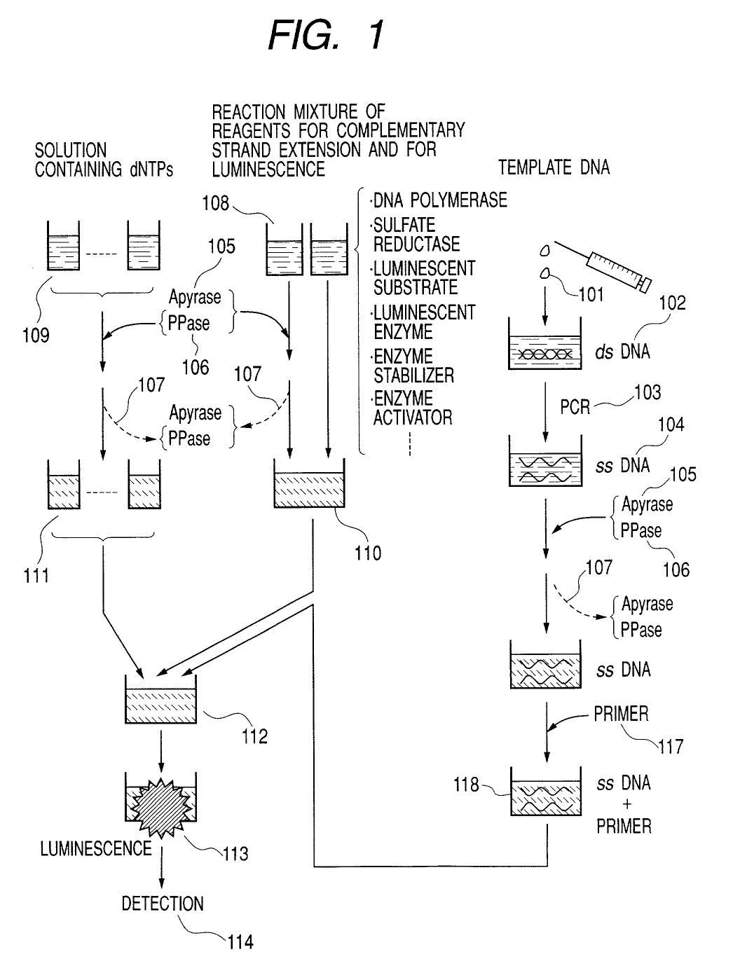

FIG. 1 is a flow chart for describing a method of preparing a noise-reduced template solution in the analysis of DNA sequence by detecting PPi, which has been generated by complementary strand extension, by chemiluminescence-reaction, in Embodiment 1 of the present invention. For preparation of a template solution of nucleic acid (DNA, RNA), DNA 102 (double stranded DNA) is extracted from the blood 101 which is a raw material for template DNA. The DNA 102 having a necessary region thereof amplified with PCR 103 or the like is used as a template. After amplification, the template is converted to a single stranded DNA 104 by using magnetic beads or the like. In a solution containing the single stranded DNA 104, dNTPs used in the amplification reaction or PPi generated by amplification reaction sometimes remains without being completely removed. The solution containing the single stranded DNA 104 is then pretreated by adding thereto apyrase 105 and PPase 106, whereby dNTPs and PPi remaining in the solution are degraded. These enzymes such as Apyrase 105 and PPase 106 will disturb the subsequent measurement so that they are removed from the solution (107). To a solution which contains the single stranded DNA 104 and from which the enzymes such as APyrase 105 and PPase 106 have been removed was added primer 117. single stranded DNA 104 and primer 117 are mixed to yield Template DNA solution 118 having primer 117 hybridized to single stranded DNA 104.

Mixed solution 108 provided for complementary strand extension reaction and chemiluminescence-reaction is a reagent kit containing DNA polymerase, sulfate reductase, luminescent substrate, luminescent enzyme, enzyme stabilizer and enzyme activator. ATP and PPi contained in Mixed solution 108 will be a cause of a noise so that they are degraded in advance by treating with enzymes such as Apyrase 105 ad PPase 106. In this case, DNA polymerase and sulfate reductase, among them, contain relatively much ATP and PPi which will be a cause for noise so that the pretreatment may be conducted only for them after separated from the others. A solution 109 containing a nucleic acid substrate for complementary strand extension reaction contains much PPi generated by thermal degradation of the nucleic acid substrate (dNTPs) and therefore becomes a cause of a noise. solution 109 containing a nucleic acid substrate is therefore reacted with enzymes, that is, Apyrase 105 and PPase 106 as a pretreatment for degrading PPi contained in solution 109. Apyrase 105 and PPase 106 used for the pretreatment are removed (107) from solutions 108 and 109 as in the preparation of the DNA temperate solution, because they disturb the subsequent measurement. These pretreated solutions 110 and 111 are provided for analysis of DNA sequence.

Extension reaction 112 is conducted in a mixture of solution 118 containing primer 117 hybridized to single stranded DNA 104, solution 110 obtained by treating a reagent kit solution 108 with enzymes Apyrase 105 and PPase 106, and solution 111 obtained by treating solution 109 containing a nucleic acid substrate with enzymes Apyrase 105 and PPase 106. PPi generated by the extension of primer 117 is detected as luminescence 113 due to chemiluminescence-reaction (114).

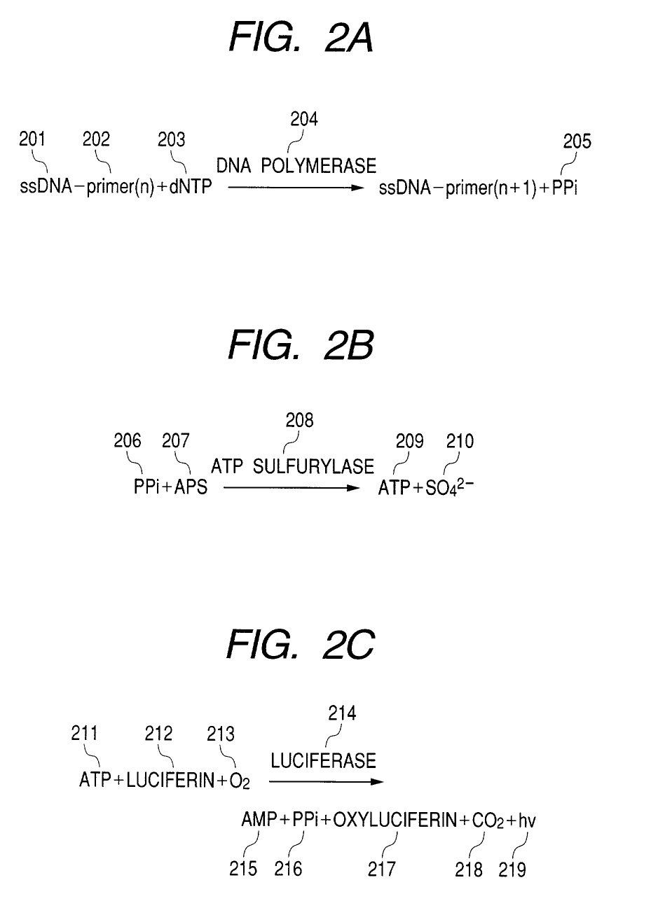

FIGS. 2A to 2C each illustrates a detection principle of PPi, which has been generated by single nucleotide extension of a primer, by chemiluminescence-reaction in Embodiment 1 of the present invention. Analysis is conducted while single stranded DNA template 201 is charged in a titer plate or the like. It is the common practice to simultaneously charge various templates in the wells of the titer plate and simultaneously conduct analysis. To the wells of the titer plate are charged single stranded DNA template 201, primer 202, dNTPs 203 as a nucleic acid substrate, DNA polymerase 204, APS (adenosine 5′-phosphosulfate) 207, ATP sulfurylase 208, Luciferin 212 and Luciferase 214 and the like.

FIG. 2A illustrates a chemical reaction wherein primer 202 hybridized to single stranded DNA 201 undergoes single nucleotide extension, thereby forming PPi 205. FIG. 2B illustrates a chemical reaction wherein PPi 205 (reference number 206 in FIG. 2B) generated by the chemical reaction of FIG. 2A reacts with APS 207 by the action of ATP sulfurylase 208, thereby forming ATP 209 and sulfuric acid ion 210. FIG. 2C illustrates a reaction wherein ATP 209 (reference number 211 in FIG. 2C) generated by the chemical reaction of FIG. 2B reacts in the presence of Luciferin 212, Oxygen 213 and Luciferase 214, thereby forming Adenosine 5′-monophosphate (which will hereinafter be abbreviated as “AMP”) 215, PPi 216, Oxyluciferin 217, Carbon dioxide 218 and one Photon (hv) 219.

Luciferin 212 is excited by free energy upon oxidation with Oxygen molecule 213 in the presence of Luciferase 214, emits a visible light and returns to a normal state. Oxidation of one molecule of Luciferin 212, emits one Photon (hv) 219 as chemiluminescence. Introduction of dNTps 203 into the DNA strand and extension of a complementary strand can be confirmed by measuring the intensity of this chemiluminescence.

PPi 216 produced by the chemical reaction of FIG. 2C reacts with APS 207 again as PPI 206 in the chemical reaction of FIG. 2B, thereby forming ATP 209. As a result, chemical reaction of FIG. 2C occurs to produce Photon (hv) 219. Described specifically, the chemical reaction of FIG. 2A introduces one Nucleic acid substrate into the DNA strand, leading to the formation of one molecule of PPi 205. This results in repetitive occurrence of the chemical reactions of FIGS. 2B and 2C and the reaction emitting Photon 219 is repeated. By introduction of one nucleic acid substrate, a large number of photons are emitted. The chemiluminescence-reactions in FIGS. 2A to 2C do not need a light source and they can provide highly sensitive and noise-free signals.

As described above, ATP and PPi contained as impurities in various reagents to be used in the chemiluminescence-reactions of FIGS. 2A to 2C are causative of noises. As described in FIG. 1, pretreatment of various reagents and a reaction solution is conducted in Embodiment 1.

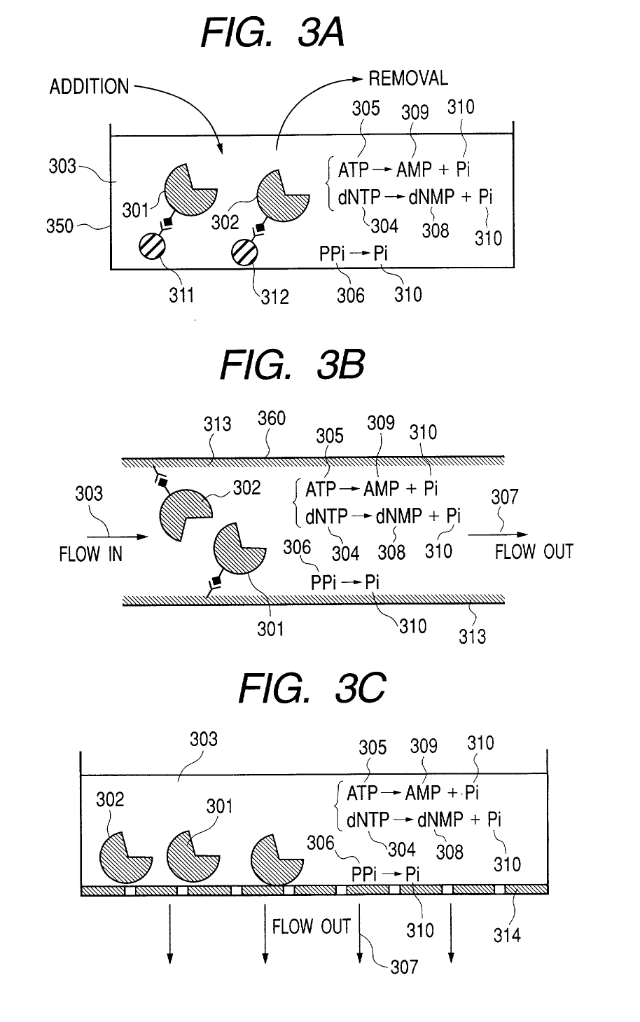

FIGS. 3A to 3C each schematically illustrates examples of the method and apparatus of pretreatment, in Embodiment 1 of the present invention, wherein impurities contained in a reagent (Mixed reagent solution 108 necessary for complementary strand extension reaction and chemiluminescence-reaction, dNTPs-containing solution 109) and Template DNA 104 are degraded with PPases and/or apyrase by using a solid phase or membrane.

In the pretreatment example of FIG. 3A, by adding a reagent kit containing Solid 311 such as beads having apyrase immobilized thereon and another reagent kit containing Solid 312 such as beads having PPase 302 immobilized thereon to Container 350 containing Reagent 108 or 109, or Template DNA 118, impurities contained in solution 303 containing Reagent 108 or 109, or Template DNA 118 are degraded, followed by removal.

The impurity ATP 305 is degraded into AMP 309 and Inorganic phosphoric acid (which will hereinafter be abbreviated as “Pi”) 310. The impurity dNTP 304 is degraded into deoxynucleotide monophosphate (dNMP) 308 and Pi 310. The impurity PPi 306 is degraded into Pi 310. From Container 350, Solids 311 and 312 are removed, whereby pretreatment is completed.

In the above-described example, a reagent kit containing Solid 311 having Apyrase 301 immobilized thereon and a reagent kit containing Solid 312 having PPase 302 immobilized thereon are used, but a reagent kit containing a solid having Apyrase 301 and PPase 302 immobilized thereon is also usable.

It is also possible to add respective inhibitors for apyrase and PPase to Container 350 after pretreatment instead of removal of Solids 311 and 312 from Container 350.

In the pretreatment example of FIG. 3B, Apyrase 301 and PPase 302 are immobilized, in advance, on Solid phase surface 313 inside or on an inner wall of the pipette tip of Tubule 360 for feeding a reaction container or template container with solution 303 containing Reagent 108 or 109 or Template DNA 104. Untreated solution 303 containing reagent 108 or 109 or Template DNA 104 is caused to flow inside of the tubule. While solution 303 containing reagent 108 or 109 or Template DNA 104 flows down through the inside of the tubule, the pretreatment as in FIG. 3A is conducted to degrade impurities contained in solution 303 containing reagent 108 or 109 or Template DNA 104. solution 307 after pretreatment is then fed to the reaction container or template container. The term “tubule” as used herein means a so-called capillary tube and a delivery passage of solution 303 is filled with a plurality of the tubules bundled to fit the inner diameter of this passage.

In the above-described example, both of Apyrase 301 and PPase 302 are immobilized on Solid phase surface 313, but either one may be immobilized.

In the pretreatment example of FIG. 3C, Apyrase 301 and PPase 302 are added to Container 370 having therein solution 303 containing un-pretreated Reagent 109 which contains at least one dNTP or analogue thereof to conduct pretreatment as described in FIG. 3A, whereby impurities PPi and ATP contained in solution 303 containing Reagent 109 are degraded. Then, Pretreated solution 307 is collected through Molecular weight selective membrane filter 314. Since dNTPs has a mean molecular weight of about 570, use of a membrane filter having NWML (Nominal molecular weight limit in Daltons) of 10,000 makes it possible to completely separate dNTPs from PPase (MW=70,000) and Apyrase (MW=50,000). In the above-described example, both of Apyrase 301 and PPase 302 are added to Container 350 having, therein, un-pretreated solution 303 containing Reagent 108 or 109 or Template DNA 104, but either one may be added.

In FIGS. 3A to 3C, pretreatment of un-pretreated solution 303 containing Reagent 108 or 109 or Template DNA 104 was described. It is needless to say that for reagents for causing chemical reaction as described in FIGS. 2A to 2C such as analogue of dNTPs, primer, APS, ATP sulfurylase, luciferin and luciferase may be pretreated similarly.

In Embodiment 1, four reagents, that is, a reagent kit containing PPase and dATPαS (dATP analogue) (deoxyadenosine 5′-triphosphate α-sulfate), a reagent kit containing PPase and dTTP (deoxythiamine 5′-triphosphate), a reagent kit containing PPase and dGTP (deoxyguanosine 5′-triphosphate, and a reagent kit containing PPase and dCTP (deoxycytidine 5′-triphosphate) are employed. In these four kits, PPase may be immobilized onto a solid such as beads.

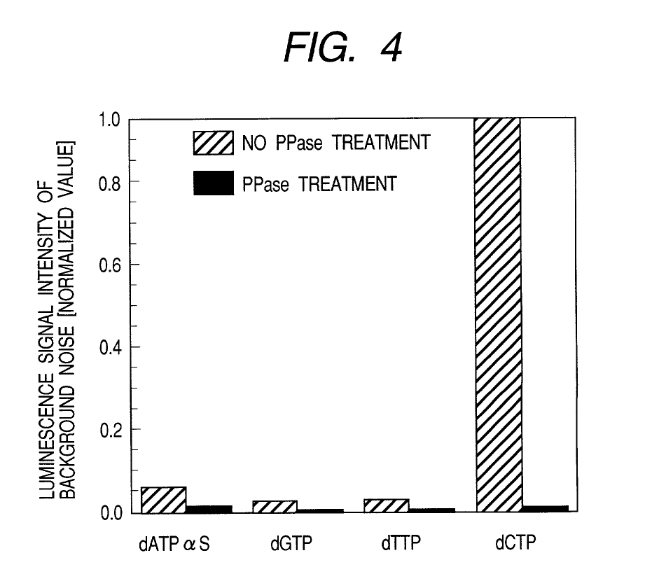

FIG. 4 illustrates comparison of noise signals between presence and absence of pretreatment with PPase in Embodiment 1 of the present invention. Among reagents used in the present invention, four dNTPs (FIG. 1: 109, FIG. 2A: 203) generate PPi by thermal degradation or the like and become the largest noise signal source. Depending on the company from which a reagent was purchased, manufacturing method, lot and storage conditions, the amount of PPI contained as impurities in four dNTPs or analogues thereof differs.

Results as shown in FIG. 4 are obtained under the following conditions. With regards to two lots of dATPαS purchased from one company, 4 lots of dGTP purchased from 3 companies, 1 lot of dTTP purchased from each of two companies-and 4 lots of dCTP purchased from 3 companies, indicated is an average of luminescence signal intensity of background noise detected by the chemiluminescence-reaction of FIGS. 2B and 2C under the conditions not permitting extension of a primer. The ordinate scale of FIG. 4 is normalized to give the maximum value of the detected noise signal of 1.0.

As shown by the results of FIG. 4, even if the content of PPi in the reagent differs, most of PPi is degraded by PPase and the luminescence signal intensity of background noise becomes sufficiently negligible. Particularly in the case of dCTP, the luminescence signal intensity of background noise is reduced to about 1/80 of that after pretreatment with PPase.

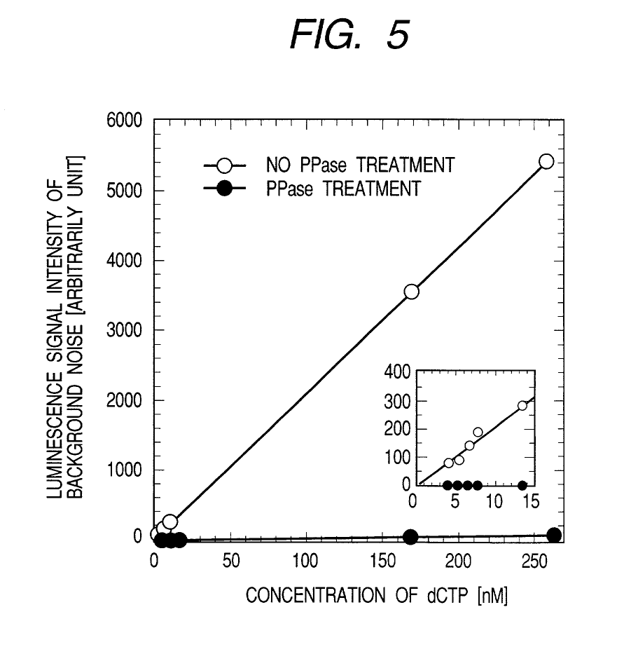

FIG. 5 illustrates dependence of noise signals on the concentration of dCTP after pretreatment with PPase in Embodiment 1 of the present invention. The content of PPi as impurities varies with the concentration of DNTPs used. Within a concentration range of from 4 nM to 260 nM, PPi contained as impurities can be degraded to a negligible level by the pretreatment with PPase. The small graph in FIG. 5 illustrates, in an enlarged from, the data in the vicinity of origin. Although measurement is not conducted so many times as indicated in the small graph, measurement results are a little exaggerated in this small graph in order to give a clear understanding of the data.

The results of FIGS. 4 and 5 suggest that use of a PPase-containing reagent kit makes it possible to degrade the impurity PPI, thereby reducing the noise signal to a negligible level in practical use.

The reagent kit to be used in the present invention is formed of respective reagents (enzyme, substrate, stabilizer, activator and the like) contained in Mixed solution 108, and, added thereto, apyrase and/or PPase as is or immobilized on a solid.

As described above, markedly high sensitivity can be attained by degradation of impurities ATP and/or PPi which will be a cause of noise signals. For example, 300 fmol of detection can be attained by detection of DNA strands accompanied by 200 bases of complementary strand extension.

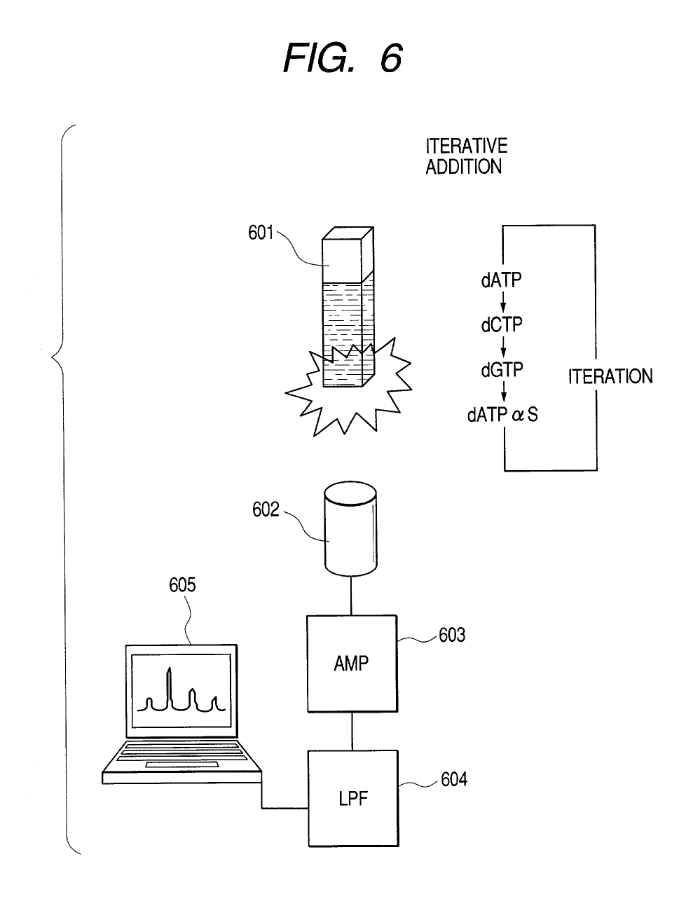

FIG. 6 is a schematic view of the constitution of a DNA sequencer employed for pyrosequencing, in Embodiment 1 of the present invention. In pyrosequencing, as illustrated in FIG. 6, four substrates, that is, dATPαS, dTTP, dGTP and dCTP are successively poured into a reaction solution in Reaction chamber 1 one by one upon complementary strand extension reaction. When the substrate thus poured is utilized by the complementary strand extension reaction, PPi is emitted to cause luminescence. The luminescence intensity is detected by Photo sensor 602 and amplified by Amplifier 603. After current-voltage conversion, it is caused to pass through Lowpass filter 604 to remove noises from the outside of the measurement system, downloaded in Data processor 605, processed, presented and stored. Whenever one base (substrate) is introduced in a DNA strand by complementary strand extension reaction, detection of the luminescence intensity is iterated.

The dNTPs remaining in the reaction solution to be detected for the luminescence intensity has a large influence on studying whether a nucleic acid substrate (base) to be added next is incorporated or not. When apyrase capable of degrading dNTPs or ATP is added to a reaction solution in advance in order to completely degrade this dNTPs or ATP after a predetermined time (within 10 seconds), ATP is consumed completely owing to competition between chemiluminescence-reaction by luciferase (FIG. 2C) and degradation reaction by apyrase (FIGS. 3A to 3C).

In pyrosequencing, PPis generated by single nucleotide extension are successively detected by chemiluminescence-reaction. The throughput of sequencing is improved by causing apyrase to exist in a reaction solution, thereby shortening the duration of luminescence (Science, 281, 363-364(1998)).

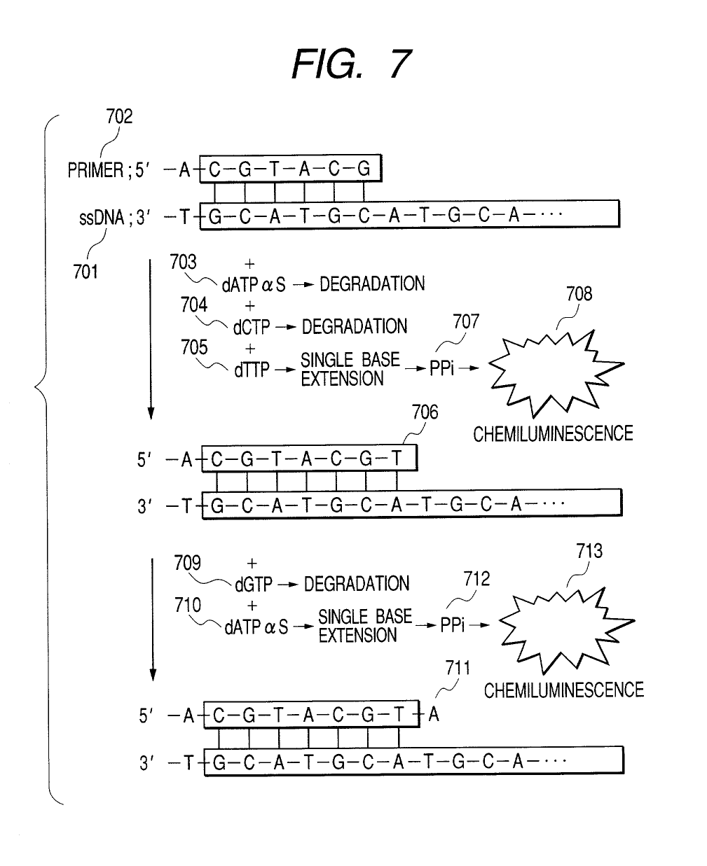

FIG. 7 illustrates a detection example of an extended strand by pyrosequencing, in Embodiment 1 of the present invention. Existence of luminescence due to chemiluminescence-reaction is studied by successively adding nucleic acid substrate dNTPs to a reaction solution containing single stranded DNA template 701 having primer 702 hybridized thereto. In the example of FIG. 7, dATPαS 703 is added first. In this case, the substrate A and target DNA strand are not complementary so that complementary strand extension reaction does not occur. Since the nucleic acid substrate is not introduced into the DNA strand, PPi is not generated and luminescence does not occur. dATPαS 703 existing in the reaction solution is degraded by apyrase existing in the reaction solution. dCTP 704 added next is not complementary to the target DNA strand so that no extension reaction occurs. Similar to dATPαS 703, dCTP 704 is degraded by apyrase. When dTTP 705 is added to the reaction solution, it is complementary to the target DNA strand so that extended strand 706 is formed, PPi 707 is generated and luminescence 708 occurs. The remaining dTTP 705 is degraded by apyrase. When dGTP 709 is added to the reaction solution, no extended strand is formed and dGTP 709 is degraded by apyrase. By the addition of dATPαS 710, Extended strand 711 is formed, PPi 712 is generated and luminescence 713 occurs.

A reagent containing nucleic acid substrates (dATPαS, dCTP, dGTP, dTTP) has PPi as an impurity and it becomes a cause of noise signals. In the present invention, PPase or a solid having PPase immobilized thereon is added to every reagent prior to pyrosequencing to degrade PPi contained as an impurity in each reagent. To the reagents other than dNTP, apyrase or a solid having apyrase immobilized thereon is added in advance to degrade the impurity ATP contained in them.

Alternatively, in pyrosequencing of the present invention, a reagent kit to which PPase or a solid having PPase immobilized thereon has been added is used. Any reagent to be used in the pyrosequencing, a reagent kit added with PPase or a solid having PPase immobilized thereon is provided. Any reagent other than dNTPs, a reagent kit added with apyrase or a solid having apyrase immobilized thereon is provided.

At least one of four nucleic acid substrates (dNTPs) 203 as shown in FIG. 2A can be replaced with its analogue, for example, dATP can be replaced with its analogue dATPαS.

Use of the reagent kit of the present invention makes it possible to degrade impurities PPi and ATP contained in the reagents, bringing about improvements in measurement sensitivity and reliability of measurement.

Embodiment II

Embodiment 2 relates to analysis of SNPs. In analysis of SNPs, typing to study existence of a mutant of each of template DNAs and measurement of a ratio of specific mutation in a mixed genome sample and its expression frequency are important. SNP is single nucleotide mutation of nucleic acid and it occurs at a frequency of several % in human population. Detailed study of single nucleotide mutation reveals mutation appearing at high frequency in those suffering from migraine or mutation appearing at high frequency in those susceptible to cancer by smoking.

SNPs related to various diseases or sensitivity to pharmaceuticals have a significant meaning in medical diagnosis and therefore, such SNPs are now being searched. Measurement of each of a vast number of SNPs obtained from the samples of a large number of people requires much labor and is not efficient.

Under such situations, a method of mixing DNA (such mixed DNA is called “pooled template”) collected from a large number of people and finding an existence ratio of mutation contained in it has recently drawn attentions. A mean existence ratio of mutation in human DNA is about 5% and about 1 to 2% of the mutation frequency is related to diseases or sensitivity to pharmaceuticals. This suggests that measurement capable of distinguishing a mean ratio of 5% from that of 6% is necessary. Such a measuring method or apparatus capable of distinguishing mean mutation frequency of 5% from that of 6%, however, has not been materialized yet and it has been an important theme. The method and reagent kit according to the present invention are also effective for this theme.

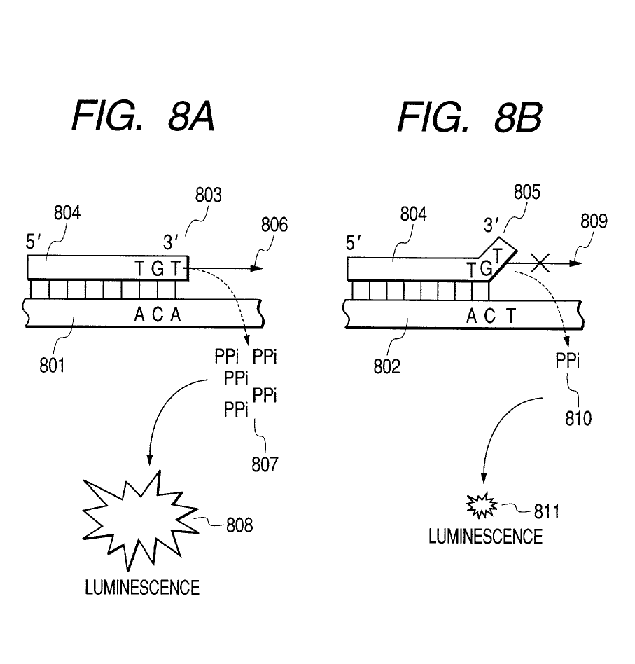

FIGS. 8A and 8B are each a schematic view for describing a principle of a measuring method of SNPs expression frequency by the primer extension method, in Embodiment 2 of the present invention. Analysis of the SNPs expression frequency (Japanese Patent Application No. 2000-300577) by the primer extension method will next be described.

As illustrated in FIG. 8A, primer 804 is prepared so that at one mutation site of single stranded DNA template 801, the 3′ terminus 803 of primer 804 coincides with a base site having SNPs. Only when the terminus base of primer 804 is complementary to single stranded DNA template 801, Complementary strand extension 806 occurs, leading to generation of a large amount of PPi 807 and occurrence of the corresponding amount of luminescence 808. As illustrated in FIG. 8B, when single stranded DNA template 802 is not complementary to 3′ Terminus base 805 of primer 804, complementary strand extension does not occur or if any, occurs slightly. Generation amount of PPi 810 and also that of luminescence 811 are therefore small.

By changing the kind of the base at the 3′ terminus of a primer, it is possible to impart the primer with a role of switch capable of causing or not causing complementary strand extension reaction (Kwok, S., et al., Nucleic Acids Res., 18, 999-1005(1990), Huang, M. M., Arnheim, N., Goodman, M. R., Nucleic acids Res., 20, 4567-73(1992)).

It is possible to improve detection accuracy of a mutation site by preparing two primers, that is, a primer complementary to a wild type template DNA and a primer complementary to a mutant-type template DNA. In pyrosequencing, luminescence due to PPi generated by one nucleotide extension is detected, while primer extension utilizes chemiluminescence-reaction due to a large number of PPis generated by multiple nucleotide extension (formation of a longer complementary strand). Since in the latter method, the intensity of light becomes stronger by about 2 figures compared with the former method and in addition, chemiluminescence-reaction continues, markedly high-sensitivity detection can be expected from this method.

With a view to heightening sensitivity further, extension reaction owing to mismatch which slightly occurs when a primer and a single stranded DNA template are not complementary must be suppressed. There is an SNPs expression frequency analyzing method (BAMPER method) wherein switching characteristics of a primer for causing or not causing complementary strand extension reaction are ensured by the use of an artificial mismatched primer which is designed to adopt a nucleic acid base not complementary to a single stranded DNA template as the third base of the primer from its 3′ terminus.

In this BAMBER method, luminescence is detected by continuously conducting complementary strand extension of several hundreds of bases, thereby generating a large amount of PPi so that this method is accompanied with such advantages as high detection sensitivity and applicability to a trace amount of single stranded DNA template and also to detection of mutation. At least one of four nucleic acid substrates (dNTPs) 203 may be replaced with an analogue thereof as illustrated in FIGS. 2. For example, dATP may be replaced with dATPαS.

Since the luminescence continues by the cyclic reaction as illustrated in FIGS. 2B and 2C, it is possible to carry out reaction on a titer plate outside the luminescence measurement system and placed therein upon measurement of luminescence. This makes it possible to measure, within one minute, chemiluminescence from 96 templates or 38 templates maintained on a titer place, and to measure a large number of titer plates successively in a short time. Thus, a markedly high measurement throughput can be accomplished. For example, supposing that chemiluminescence of 96 templates is measured at 1 minute intervals and this measurement is conducted for 10 hours a day, about 60000 templates can be measured in one day.

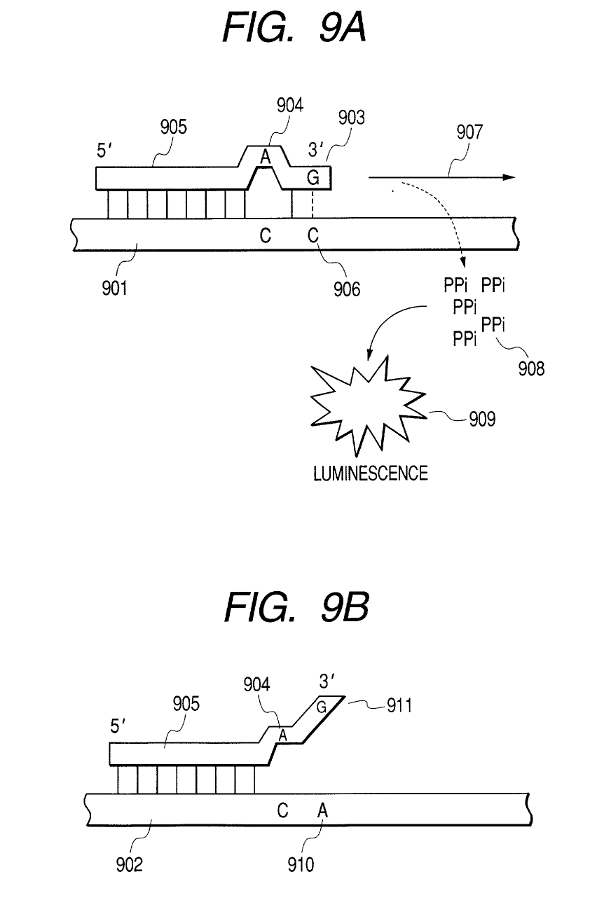

FIGS. 9A and 9B each illustrates a principle of a high sensitivity measuring method (BAMPER method) of SNPs expression frequency based on the primer extension method using an artificial mismatched primer, in Embodiment 2 of the present invention.

First, prepared is single stranded DNA template 901 hybridized to PCR or specific DNA probe and regulated by complementary strand extension reaction. This single stranded DNA template 901 is regulated as illustrated in FIG. 9A to have base C, which is Nucleic acid base 906, as one SNPs.

Then, artificial mismatched primer 905 is prepared. primer 905 is adjusted so that base G of 3′ terminus 903 is complementary to Nucleic acid base 906 of Template DNA 901 and the third Nucleic acid base 904 from the 3′ terminus is base A not complementary to Template DNA 901. Primer 905 and others are adjusted to have a sequence complementary to that of DNA template 901.

In single stranded DNA template 901 and artificial mismatched primer 905 thus prepared, base G at the 3′ terminus of artificial mismatched primer 905 is complementary to base C 906 of single stranded DNA template as illustrated in FIG. 9A, so that complementary strand extension 907 proceeds, a large amount of PPi 908 is generated and luminescence 909 occurs. By this, existence of one SNPs per single stranded DNA plate 901 is confirmed.

Separately, single stranded DNA template 902 is prepared in a similar manner to that of single stranded DNA template 901. As illustrated in FIG. 9B, this single stranded DNA template 902 is adjusted to have base A 910 at a site, wherein one SNPs exists in the case of single stranded DNA template 901, instead of base C 906.

When hybridization of single stranded DNA template 902 to artificial mismatched primer 905 is intended, 3′ terminus 911 of primer 905, on the contrary, widens the distance from single stranded DNA template 902 because base G at the 3′ terminus of primer 905 is not complementary to base A at Site 910 of single stranded DNA template 902. Complementary strand extension therefore does not occur, generation of PPi as illustrated in FIG. 9A hardly occurs, leading to almost no generation of luminescence.

As described above, the kind of the second or third base from the 3′ terminus of artificial mismatched primer 905 is not so influential to the progress of complementary strand extension reaction. When base 911 at the 3′ terminus of artificial mismatched primer 905 is not complementary to base 910 of single stranded DNA template 902, on the other hand, the 3′ terminus of artificial mismatched primer 905 is almost completely apart from the single stranded DNA template and complementary stranded extension reaction due to mismatch substantially stops its progress. This clarifies presence or absence of SNPs and improves detection sensitivity.

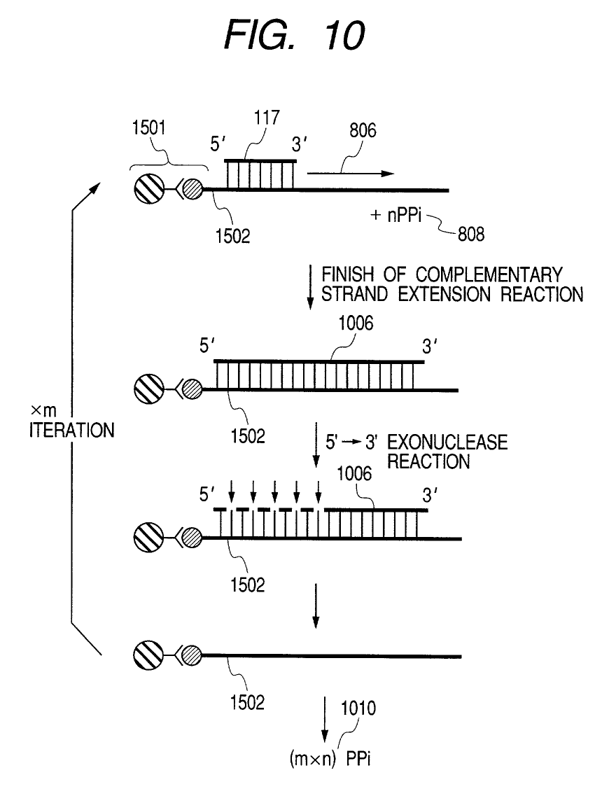

FIG. 10 illustrates a principle of high sensitivity measurement of SNPs expression frequency by the primer extension method using an artificial mismatched primer and 5′ → 3′exonuclease, in Embodiment 2 of the present invention. In this method, enzymatic cleaving of an extended complementary strand, followed by iteration of complementary strand extension reaction makes it possible to generate a vast molecular number of pyrophosphoric acid, thereby improving detection sensitivity (refer to Japanese Patent Application 2000-300577).

As illustrated in the top diagram, Target single stranded DNA 1502 having 5′ terminus thereof immobilized on a solid 1501 and primer 117 are, together with DNA polymerase, placed in a reaction solution. As a result, as described in FIG. 9A, Extended complementary strand 806 of primer 117 and also PPi 808 are formed. In this example, however, enzymes relating to luminescence are not charged in the reaction solution at this extension stage of Complementary strand 806 of primer 117.

This complementary strand extension reaction ends with completion of a predetermined extension of Complementary strand 806, whereby double stranded DNA 1502 having Complementary strain 1006 formed by Extended complementary strand 806 of primer 117 and Target DNA 1502 is formed. Here, exonuclease for causing enzymatic cleavage of the complementary strand is charged in the reaction solution. As a result, Complementary strand 1006 thus formed is cut into nucleotides by exonuclease reaction, whereby Target single stranded DNA 1502 is regenerated.

A description will next be made of exonuclease for causing enzymatic cleavage of a complementary strand. Exonuclease can be classified into 5′ → 3′exonuclease having activity of degrading the DNA strand from the 5′ terminus to the 3′ terminus and 3′ → 5′exonuclease having activity of degrading the DNA strand from the 3′ terminus. For utilization of match or mismatch of the 3′ terminus of a primer to detect synthesis of a complementary strand, 3′ → 5′exonuclease is not suited. This enzyme etches a mismatched site of the primer, preventing differentiation between normal DNA and DNA having mutation. The 5′ → 3′exonuclease degrades the DNA strand of the double stranded DNA from its 51 terminus, but by immobilizing the 5′ terminus of Target DNA 1502 on Solid 1501 such as beads or modifying the 5′ terminus, the target DNA is protected in advance from degradation by 5′ → 3′exonuclease. For finding mutation by mismatch at the 3′ terminus, 5′ → 3′exonuclease is suited.

Excessive primer 117 in the reaction solution is hybridized again to Target DNA 1502 to form Extended complementary strand 1006 of primer 117, and this generates PPi 808 newly.

Formation of Extended complementary strand 806 of primer 117 is followed by formation of (n) pieces of molecules of PPi 808, this (n) corresponding to the number of extended bases. Supposing that formation of Extended complementary strand 806 and degradation of Complementary strand 1006 by the 5′ → 3′exonuclease reaction are repeated m times, Extended complementary chain 806 is formed (m) times in total, leading to generation of (m×n) molecules of PPi 1010.

When enzymes relating to luminescence are poured into the reaction solution after generation of (m×n) molecules of PPi 1010, PPi 1010 is converted into ATP and ATP oxidizes luciferin, leading to formation of PPi. ATP luciferase acts on the PPi formed newly and converts it into ATP again, and this ATP reacts with luciferin. At m=10, about 1000 PPis are released upon formation of one DNA complementary strand. This is repeated 10 times, by which the luminescence intensity becomes about 10000 times as much as that obtained by pyrosequencing.

Even in this example, complementary strand extension reaction does not proceed upon hybridization of primer 117 to Target DNA 1502 when primer 117 has mismatch at the 3′ terminus thereof as illustrated in FIG. 9B. Mutation can be studied accurately if sequencing is devised properly to permit disposal of artificial mismatch in the vicinity of the 3′terminus of primer 117 as needed.

In either one of the primer extension method as described in FIGS. 8A to 8B or the BAMPER method as described in FIGS. 9A to 9B and FIG. 10, reagents to be used contain, as impurities, ATP and PPi, which are causative of noise signals. In analysis of SNPs expression frequency as described in FIGS. 8A and 8B, and FIGS. 9A and 9B, the present invention makes it possible to drastically reduce noise signals and improve detection sensitivity.

As in Embodiment 1, prior to analysis of SNPs expression frequency as illustrated in FIGS. 8A and 8B and FIGS. 9A and 9B, PPase or a solid having PPase immobilized thereon is added to every reagent to degrade PPi contained as impurities in it, while to each of the reagents other than dNTP, apyrase or a solid having apyrase immobilized thereon is added to degrade ATP contained as an impurity in it.

Alternatively, in analysis of SNPs expression frequency as illustrated in FIGS. 8A and 8B, FIGS. 9A and 9B, and FIG. 10, a reagent kit added with PPase or with a solid having PPase immobilized thereon is used. In analysis of SNPs expression frequency as illustrated in FIGS. 8A and 8B, FIGS. 9A and 9B, and FIG. 10, a reagent kit added with PPase or with a solid having PPase immobilized thereon is provided for each of the reagents used for this analysis. In addition, a reagent added with apyrase or with a solid having apyrase immobilized thereon is provided for each of the reagents other than dNTP.

By the use of the reagent kit of the present invention, impurities such as PPi and ATP contained in reagents are degraded, which brings about improvements in detection sensitivity and detection reliability.

In pyrosequencing, excessive dNTP is promptly degraded by the addition of apyrase to the reaction solution in order to prevent excessive dNTP from disturbing the subsequent reaction. In this case, apyrase degrades ATP together, whereby ATP is consumed in competition by chemiluminescence-reaction with luciferase (FIG. 3C) and degradation reaction by apyrase (FIGS. 3A to 3C). The intensity of luminescence caused by chemiluminescence-reaction is reduced to several to several tens % compared with that without APyrase.

For determination of a base sequence or SNPs detection by making use of this pyrosequencing, about 0.2 pmol of a DNA temperate is necessary.

In the present invention, high sensitivity detection is available in the BAMPER method, because after degradation of impurities by using a solid having PPase immobilized thereon and a solid having apyrase immobilized thereon, the solids are taken out from the reaction mixture, which makes it possible to efficiently react ATP, which has been generated by PPi released upon extension of a complementary strand, with a luminescent substrate such as luciferin.

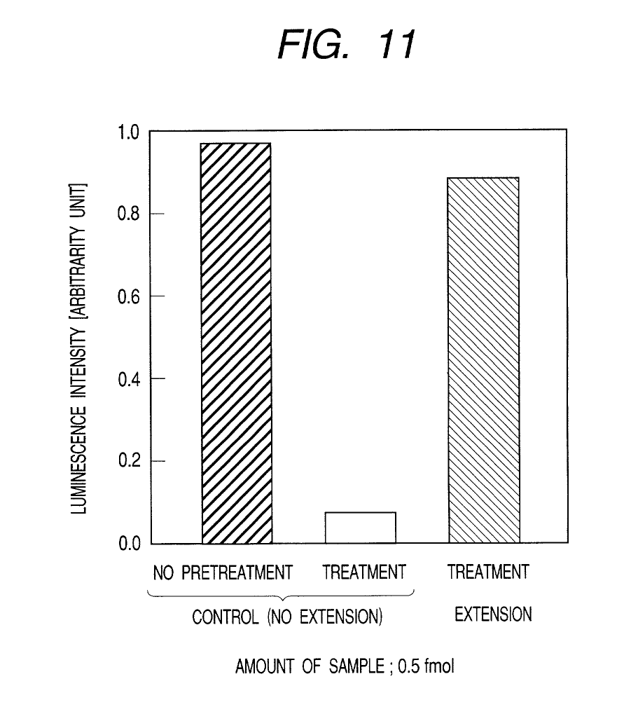

FIG. 11 illustrates an example of the effects of the pretreatment with PPase in SNPs detection by the primer extension method (BAMPER method) using an artificial mismatched primer, in Embodiment 2 of the present invention. This describes results of SNPs expression frequency analysis by the BAMPER method, depending on whether the reagent kit of the present invention is used or not.

As a template, single stranded DNA of a portion of M13mp18 having a sequence as listed in SEQ ID NO: 1) is used in an amount of 0.5 fmol. In the sequence, the underlined base corresponds to an SNPs site. A primer having a sequence of SEQ ID NO: 2) is used as an artificial mismatched primer whose 3′ terminus coincides with the SNPs site (permits extension of a complementary strand), while a primer having a sequence of SEQ ID NO: 3) is used as an artificial mismatched primer whose 3′ terminus does not coincide with the SNPs site (does not permit extension of a complementary strand). In the sequences of SEQ ID NO: 1), SEQ ID NO: 2) and SEQ ID NO: 3), the mismatched sites are indicated by bases in capital letters.

| | |
|---|---|
| acaggaaaca gctatgacca tgattacgaa ttcgagctcg gtacccgggg | (SEQ ID NO: 1) |
| atcctctaga gtcgacctgc aggcatgcaa gcttggcact Ggccgtcgtt | |
| ttaca: | |
| tgtaaaacga cggcGag: | (SEQ ID NO: 2) |
| tgtaaaacga cggcGat: | (SEQ ID NO: 3) |

In FIG. 11, luminescence intensity when the pretreatment of the present invention is not conducted under extension-free conditions is indicated by “no extension”, while the luminescence intensity when, prior to the BAMPER method, reagents to be used for the BAMPER method are pretreated using the PPase- and APyrase-containing reagent kit of the present invention in order to degrade impurities is indicated by “pretreatment”. In the case of “no pretreatment”, luminescence intensity as noise signals is markedly high and signals resulting from presence or absence of the SNPs site cannot be separated when the amount of Template DNA is trace. In the case of “pretreatment”, on the other hand, the luminescence intensity as noise signals is very small.

In FIG. 11, the luminescence intensity caused by extension when template DNA having SNPs is analyzed using the PPase- and APyrase-containing reagent kit of the present invention in accordance with the BAMPER method is indicated as “pretreatment”, with the data under extension free conditions as a control. Comparison of the luminescence intensity between data with and without extension of a complementary strand clearly indicates the effects of the present invention.

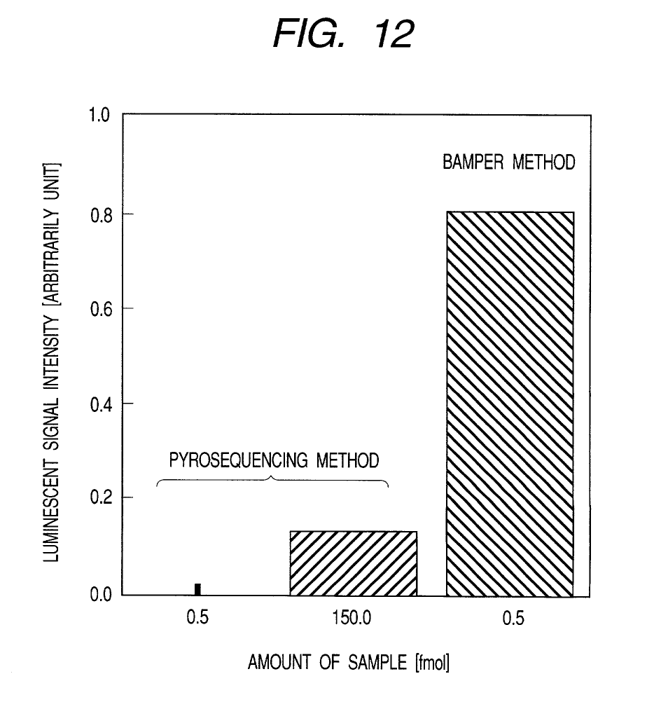

FIG. 12 illustrates comparison of sensitivity of SNPs detection between the primer extension method using an artificial mismatched primer and pyrosequencing, in Embodiment 2 of the present invention. A description will next be made of the SNPs detection results in accordance with each of the pyrosequencing method (Anal Biochemistry, 280, 103-110(2000)) and the BAMPER method, by using reagents pretreated with the PPase- and apyrase-containing reagent kit of the present invention.

As a template, single stranded DNA of a portion of M13 mp18 having the sequence of SEQ ID NO: 1 similar to that used in the analysis of FIG. 11 was used. The amount of the template was 0.5 fmol or 150.0 fmol in the pyrosequencing method, while it was 0.5 fmol in the BAMPER method. A normal primer having a sequence of SEQ ID NO: 4 was used in the pyrosequencing method, while an artificial mismatched primer having a sequence of SEQ ID NO: 2 similar to that used in the analysis of FIG. 11 was used in the BAMPER method. In the sequence of SEQ. ID. NO: 4, the mismatched site is indicated by the bases in a capital letter.

tgtaaaacga cggccag: (SEQ ID NO: 4)

The luminescent signal intensity available by the application of the present invention to the BAMPER method is much higher than that available by the conventional pyrosequencing method. When the amount of the template was 0.5 fmol, no luminescence was detected by the pyrosequencing method. When a ratio S/B (signals based on the formation of extended strands/noise signals when extended strains are not formed) is about 13 in the BAMPER method and about 14 in the pyrosequencing method, a ratio of the luminescent signal intensity detected by the former method to the latter one, that is, detection sensitivity ratio is about 2000:1.

Upon detection of SNPs by the BAMPER method, use of reagents pretreated according to the present invention enables about 2 figures to 3 figures higher sensitivity detection compared with the conventional pyrosequencing method. In the conventional pyrosequencing method or typical sequencing based on gel electrophoresis, a template of the pmol order is necessary, but the amount of a single stranded DNA template necessary for the BAMPER method to which the present invention has been applied is as less as 50 amol or smaller.

Expression frequency analysis can be carried out by preparing two primers, that is a primer complementary to a wild type single stranded DNA template and a primer complementary to a mutation-having single stranded DNA template, for detecting SNPs sites by the BAMPER method.

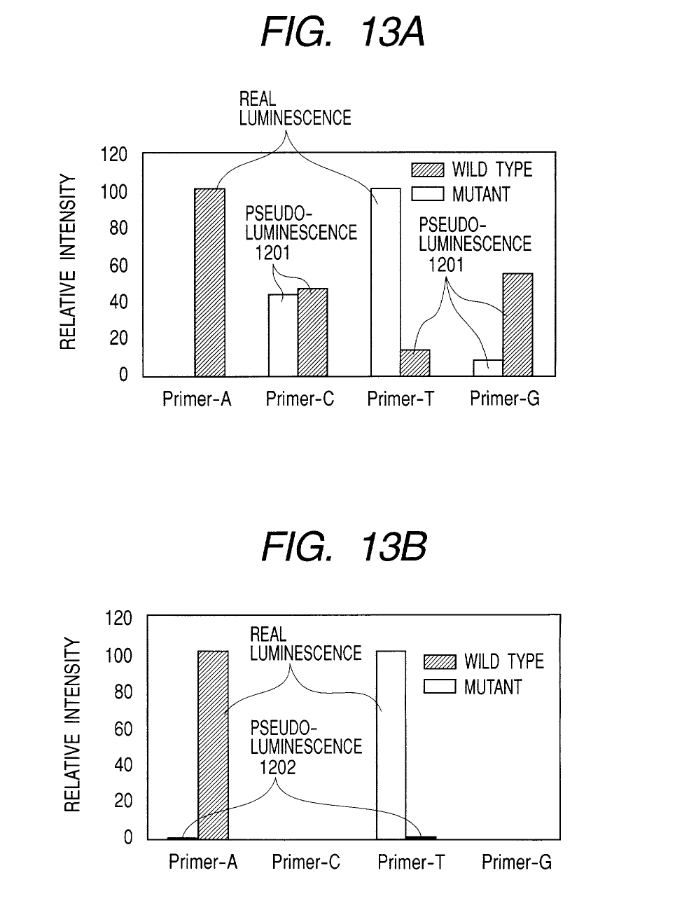

FIGS. 13A and 13B illustrate comparison between use of a conventional primer and use of an artificial mismatched primer for determining real luminescence intensity and pseudo-luminescence intensity by the measurement of SNPs expression frequency, in Embodiment 2 of the present invention. Detection results of SNPs upon use of a portion of p53 gene as a single stranded DNA template will be described below.

The wild type single stranded DNA has a sequence of SEQ ID NO: 5. Primer-A is complementary to the wild type single stranded DNA and it has a sequence of SEQ ID NO: 6. The mutant single stranded DNA has a sequence of SEQ ID NO: 7. Primer-T is complementary to the mutant single stranded DNA and has a sequence of SEQ ID NO: 8. Two ordinarily-used primers which do not originally emit luminescence are designated as primer-C having a sequencing of SEQ ID NO: 9 and primer-G having a sequence of SEQ ID NO: 10. In the sequences from SEQ ID NO: 6 to SEQ ID NO: 11, bases in capital letters are mismatched sites.

| | |
|---|---|
| ctttcttgcg gagattctct tcctctgtgc gccggtctct cccaggacag | (SEQ ID NO: 5) |
| gcacA aacacgcacc tcaaagctgt tccgt cccagtagat tacca: | |
| aacagctttg aggtgcgtgA tt: | (SEQ ID NO: 6) |
| ctttcttgcg gagattctct tcctctgtgc gccggtctct cccaggacag | (SEQ ID NO: 7) |
| gcacTaacac gcacctcaaa gctgttccgt cccagtagat tacca: | |
| aacagctttg aggtgcgtgA ta: | (SEQ ID NO: 8) |
| aacagctttg aggtgcgtgA tg: | (SEQ ID NO: 9) |
| aacagctttg aggtgcgtgA tc: | (SEQ ID NO: 10) |

As shown in FIG. 13A, when ordinarily-employed primers are used without the pretreatment of the present invention or without use of the PPase- and apyrase-containing reagent kit of the present invention, pseudo-luminescence (false-positive-luminescence) 1201 occurs owing to the reaction due to impurities or the pseudo-incorporation of a nucleic acid substrate into the DNA strand even when primer-C and primer-G which are originally free from emission of luminescence are used.

As illustrated in FIG. 13B, however, generation of pseudo-luminescence 1202 can be substantially suppressed in the BAMPER method using an artificial mismatched primer having a nucleic acid base, which is not complementary to the base of the template DNA, artificially introduced into the third base from the 3′ terminus of the primer, when the pretreatment of the present invention is conducted or the PPase- and apyrase-containing reagent kit of the present invention is used. This brings about high sensitivity detection.

When the template contains both the wild and mutant DNAs, the chemiluminescence intensities I_{W} and I_{M} due to PPi generated by the complementary strand extension reaction using two primers P_{W} and P_{M} complementary to these DNAs, respectively, are proportional to the existence ratios I_{W} of the wild DNA and I_{M} of the mutant DNA. In other words, the existence ratios A_{W} and A_{M} of the wild and mutant DNA in the template can be found by determining a relative intensity I_{W}/(I_{W}+I_{M}), and I_{M}/(I_{W}+I_{M}) based on the results of measuring the luminescence intensities I_{W} and I_{M} upon two chemiluminescence-reactions. Measurement of these existence ratios are affected by the chemiluminescence resulting from impurities contained in each reagent. As illustrated in FIGS. 13A and 13B, however, chemiluminescence due to impurities is reduced by the use of the reagent kit of the present invention so that the existence ratio can be measured at high precision.

Analysis results of SNPs expression frequency by using a p53 gene and artificial mismatched primer in Embodiment 2 of the present invention are shown in Table 1.

TABLE 1

| Existence ratio | Expression frequency |
|---|---|
| [A_{M} = I_{M}/(I_{W} + I_{M})] | [mean ± error] |
| 0.020 | 0.019 ± 0.003 |
| 0.050 | 0.051 ± 0.008 |
| 0.100 | 0.096 ± 0.008 |
| 0.700 | 0.694 ± 0.010 |

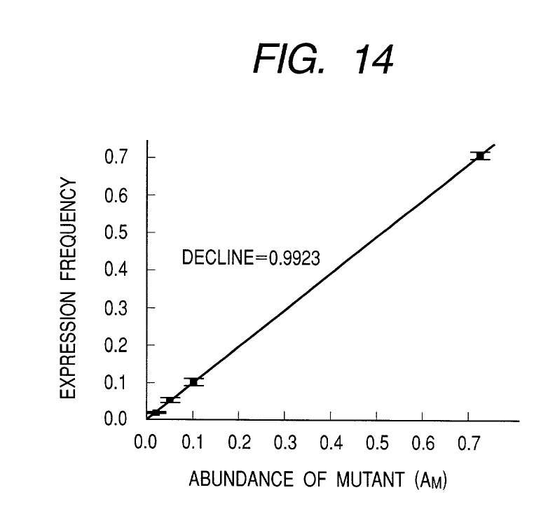

FIG. 14 is a graph illustrating analysis results of SNPs expression frequency in accordance with the BAMPER method by using an artificial mismatched primer, in Embodiment 2 of the present invention. Mean expression frequency agrees with the existence ratio (SNP allele frequency) with an error of 0.6% or less and the existence ratio can be determined with an error of 1% or less.

Simple typing (typing for determining whether the template is a wild type, mutant or hetero of wild type and mutant) is very convenient and typing can therefore be effected with substantially 100% accuracy.

EMBODIMENT III

In Embodiment 3, a plurality of single nucleotide polymorphisms (SNPs) of one template DNA are successively analyzed in one reaction vessel.

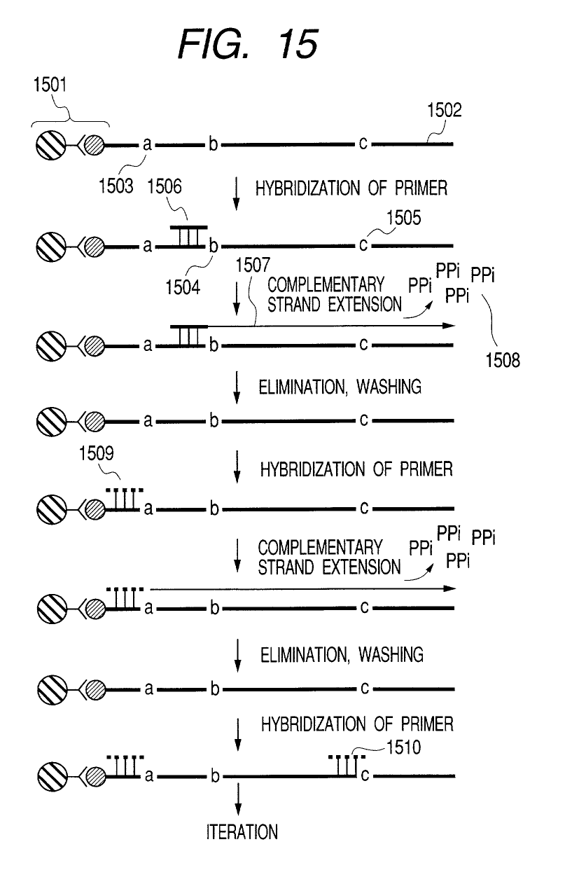

FIG. 15 illustrates a measuring method, in Embodiment 3 of the present invention, of a plurality of SNPs existing in one DNA or a plurality of DNAs by using one reaction vessel. Similar to Embodiment 2, an artificial mismatched primer for the BAMPER method is employed in this Embodiment 3. Template DNA is prepared by extracting DNA from the blood, amplifying a necessary sequence region with PCR or the like or hybridizing a specific DNA probe (different from a primer used for SNPs detection) to DNA, and carrying out complementary strand extension reaction.

When single stranded DNA is prepared using a solid such as avidin-coated magnetic beads, it is the common practice to use a biotinylated primer. At this time, the template DNA solution thus prepared contains dNTPs used in the amplifying reaction or PPi generated upon amplifying reaction so that they are degraded with an enzyme such as apyrase or PPase to markedly reduce noise signals in chemiluminescence-reaction. When these enzymes disturb the measurement of chemiluminescence, they are separated from the reaction solution and then taken out of the measurement system by using the method and apparatus as illustrated in FIGS. 3A to 3C.

Single stranded DNA template 1502 has plural SNPs sites, a 1503, b 1504, and c 1505, they can be analyzed using the same DNA. For example, the 3′ terminus of the target single stranded DNA template 1502 is immobilized on Solid 1501 such as beads, followed by hybridization of First primer 1506 whose 3′ terminus coincides with SNPs site b 1504 of single stranded DNA template 1502.

Only when the 3′ terminus base of First primer 1506 is complementary to the base at SNPs site b 1504 of DNA template 1502, complementary strand extension 1507 occurs, generating a large amount of PPi 1508, whereby a large amount of luminescence occurs by the chemiluminescence-reaction. When the 3′ terminus base of the first primer is not complementary to the base of single stranded DNA template 1502, complementary strand extension does not occur, neither does luminescence. Existence of mutation is judged by this luminescence.

After completion of the measurement of luminescence by First primer 1506, reagents are washed off. The complementary strand extended and hybridized to the single stranded DNA template is dissociated by raising the temperature of the solution, alkalizing or degrading, as described in FIG. 10, by 5′ → 3′exonuclease reaction, whereby the single stranded DNA template is regenerated in the solution.

Single stranded DNA template 1502 is hybridized to Second primer 1509 whose 3′ terminus base coincides with SNPs site a 1503 to cause extension reaction and then, luminescence caused by chemiluminescence-reaction is measured. After completion of the measurement of luminescence from single stranded DNA template 1502, only the template is left in the solution in the above-described manner.

Third primer 1510 is then hybridized to single stranded DNA template 1502 similarly, followed by extension reaction. The luminescence caused by chemiluminescence-reaction is measured. Similar operations are then repeated successively.

Alternatively, by making use of the property of PPase capable of degrading PPi but not dNTPs, it is possible to detect different SNPs sites successively without removing extended strands formed by the first primer. In this case, PPase is introduced into the reaction solution or is taken out of the reaction system as needed by using the method and apparatus as described in FIG. 3. In this case, the hybridized sites of the second and third primers may exist on the same DNA strand or different DNA strands. When they are on the same DNA strand, the second primer is disposed to the 3′ terminus side of the DNA strand than the first primer, which makes it possible to allow the complementary strand extension to proceed while removing the DNA complementary strand formed by the first primer.

In this manner, use of PPase makes it possible to reduce background noise, thereby attaining high sensitivity detection of SNPs sites of single stranded DNA template. By washing off PPi or removing PPi by degradation, the same template DNA can be used repetition as a template (target) of complementary strand extension reaction and this makes it possible to study various SNPs sites by using only one template DNA. In addition, use of PPase contributes to saving of labor for template preparation and efficient analysis. In Embodiment 3, description was made of only one DNA, but even if a plurality of template DNAs are used at the same time, analysis can be conducted easily in a similar manner.

Measurement can be conducted without the above-described washing step. In this case, after detection of luminescence, the reaction system is taken out of the measuring part, and is allowed to stand for several minutes to cause sufficient degradation of PPi by PPase, or is added with a large amount of PPase to cause prompt degradation of PPi. Thus, duration of luminescence is shortened so as not to cause problems in measurement. In this measurement method, a second primer is charged to effect complementary strand extension reaction after the luminescence intensity becomes sufficiently weak. The reaction solution contains dNTPs and DNA polymerase so that the complementary strand extension reaction proceeds without problems. Then, a large amount of PPi is generated and luminescence appears by the chemiluminescence-reaction. The reaction system is introduced into the measuring part, whereby chemiluminescence is measured.

In the above-described embodiments of the DNA sequence analysis method of the present invention, usefulness of a reagent solution treated in advance with PPase and apyrase was described. Enzymes such as PPase and apyrase exhibit activity within the acting temperature. When reagents to be used are treated with PPase, it is important to set its amount (concentration) within a range permitting sufficient degradation of impurity PPi and not affecting the signal detection. When the amount is set within a proper range, the remnant of PPase, if any, will not disturb the measurement.

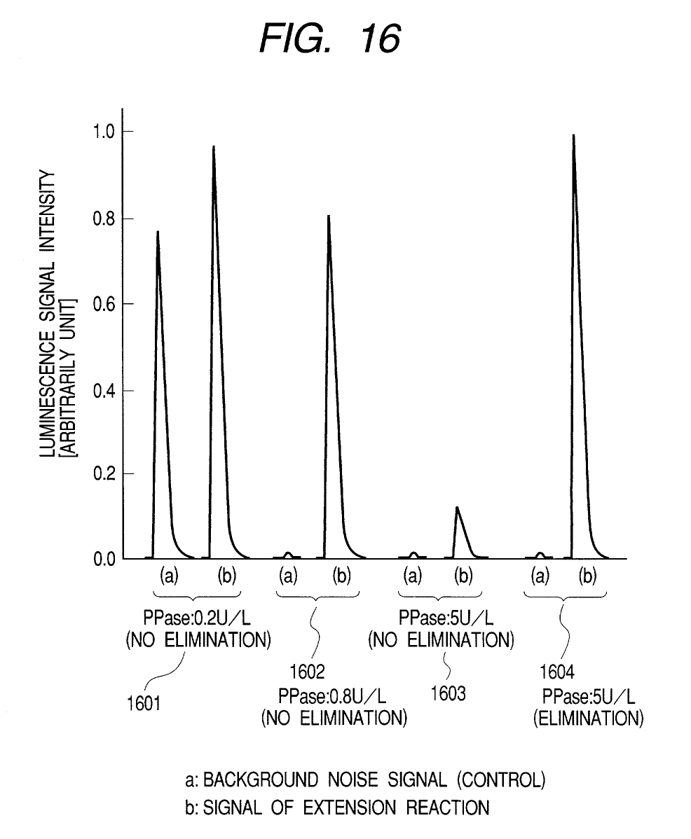

FIG. 16 illustrates comparison in signal intensity depending on the residual amount of PPase added in the pyrosequencing method, in Embodiment 1 of the present invention. Signal measurement was conducted under the conditions as shown in Table 2.

TABLE 2

| | |
|---|---|
| Amount of template DNA | 0.2 pmol (2.5 iM) |
| Treating temperature with | Room temperature |
| PPase | |
| Treating time with PPase | 30 minutes |
| Photo detector | Photomultiplier |
| Gain | 10⁶ times |
| Applied voltage | 10³ V |
| Signal amplification ratio | 10⁶ times |

As a template, single stranded DNA of a portion of p53 gene of SEQ ID NO: 11 was used.

gtggtaatct actgggacgg aacagctttg aggtgcgtgt ttgtgcctgt cctgggagag acc (SEQ ID NO: 11)

As a primer, an extension primer having a sequence of SEQ ID NO: 12 completely complementary to the template DNA was employed and signal intensities of the underlined three bases ttt in the template DNA were compared.

ggtctctccc aggacaggca (SEQ ID NO: 12)

FIG. 16 illustrates comparison in luminescence intensity between the noise signal (a), serving as a control, in the case where PPase treatment is conducted under extension free conditions, and (b) signal of extension reaction, with the residual amount as a parameter.

When the signal (S) intensity observed under the conditions permitting extension is 1.25 times or greater (S/N=1.25), it can be detected as a signal due to extension-induced luminescence. As is apparent from the diagram, DNA sequence can be analyzed by treatment of the reaction solution in advance with 0.2 U/L of PPase (1601). At this concentration, however, the noise signal level is still high, which does not permit high sensitivity detection. It is recommended to act a sufficient amount of PPase on the reaction solution until the noise signal disappears. By the preliminary treatment of the reaction solution with 5.0 U/L of PPase (1603), noise signal disappears substantially, but at the same time, signals due to extension also decrease. This occurs because prior to luminescence reaction, a rise in the degradation rate of PPi inevitably degrades PPi emitted due to extension. As a result, luminescence cannot be detected easily in spite of complementary strand extension of the nucleic acid template and in this case, high sensitivity detection cannot be attained. As is not illustrated in the diagram, as the residual amount exceeds 20 U/L or greater, the addition of PPase makes it more difficult to detect the luminescence signal. For example, the diagram suggests that the method (1604) of conducting the pretreatment with 5.0 U/L of PPase and after removal of it, measuring the luminescence provides the best S/N and enables high sensitivity. There is however not a significant difference between this method and a method (1602) of adding 0.8 U/L of PPase (1602), from the viewpoint of having a S/N providing sufficient degradation capacity for measurement.

This means in the present invention that preliminary PPase treatment is indispensable and that noise signals can be reduced sufficiently and at the same time, signals having enough luminescent intensity are available even without removal of PPase when its amount is optimum, or even after treatment with a large excess of PPase, by removing it prior to measurement. This enables high sensitivity detection.

Apyrase is added to a reagent solution other than a solution containing dNTPs or analogue thereof. The above-description also applies to apyrase.

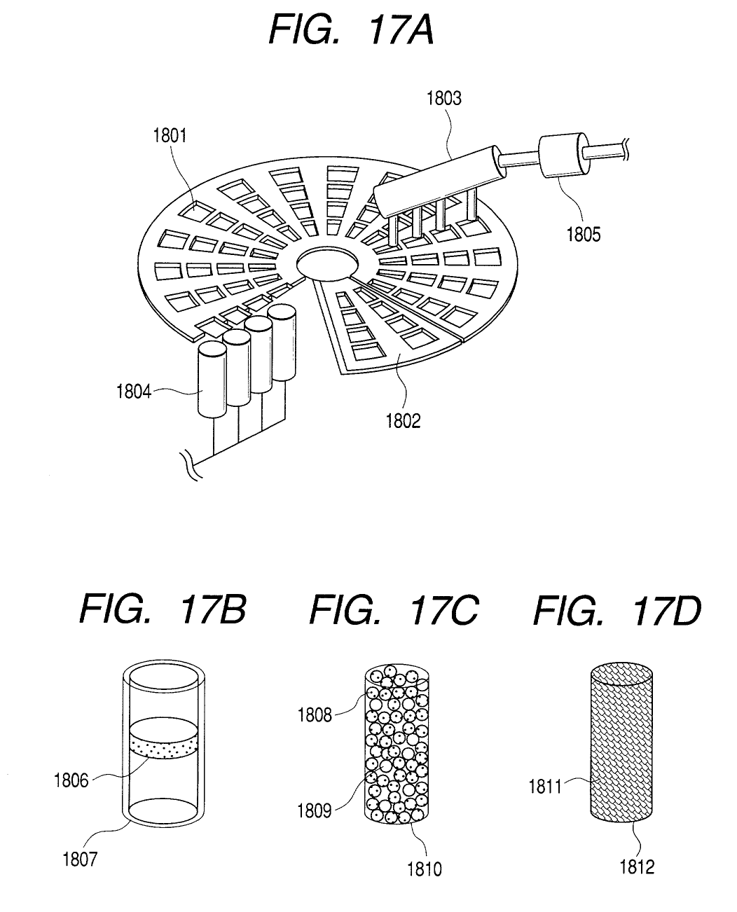

FIGS. 17A to 17D each illustrates an automated DNA sequencer of the present invention having a step of treating a reaction reagent by using PPase and apyrase, wherein FIG. 17A illustrates the whole constitution and FIGS. 17B to 17D each illustrates specific examples of the part for removing an impurity such as PPi or ATP from the reagent. The example illustrated by the diagrams suggests that this analyzer enables simultaneous analysis of a large number templates and, owing to online automation of various operations necessary for measurement such as pouring of a reagent, pouring of a template and detection, it also enables improvement in throughput of DNA sequence analysis. In these diagrams, indicated at numeral 1801 is Reaction vessel having a capacity of several μL or less and 1802 is an analysis chip. Many Reaction vessels 1801 are disposed on the concentrically on analysis chip 102. Analysis chip 1802 may be in the CD form as illustrated in this example or may be in the form of a square sheet. Indicated at numeral 1803 is Reagent dispenser for feeding Reaction vessel 1801 with a primer, reagents necessary for complementary strand extension reaction and reagents necessary for luminescence-reaction. Indicated at numeral 1805 is a removal part for removing impurities such as PPi or ATP from the reagents.

In the present invention, treatment of reagents with PPase or apyrase is conducted in prior to measurement. Removal part 1805 for removing impurities such as PPi or ATP from the reagents is disposed upstream of Reagent dispenser 1803 for feeding reagents to a template. Removal part 1805 as illustrated in FIG. 17B is, for example, formed of Filter 1806 and Filter holder 1807. When a reagent containing PPase or apyrase immobilized on a solid such as beads is employed, it is removed from the reagent through Filter 1806. Although no limitation is imposed on the material of Filter 1806 and a polymer sheet such as polystyrene or porous glass is usable, that having a diameter φ of about 1 to 2 cm and a pore size φ of about 0.2 to 1.4 μm is used. A solution containing beads of a diameter φ of about 3 μm having an enzyme immobilized thereto is fed to the filter at a flow rate of about 2 mL/min. The size of the filter holder can be selected as needed depending on the amount of the reagent to be treated, but the total length is about 1 to 5 cm. As illustrated in FIG. 3c, use of a molecular-weight-selective-membrane-filter of about NWML=10,000 instead of the above-described filter makes it possible to remove PPase molecules or apyrase molecules directly. FIG. 17C illustrates one example of Removal part 1805 in the form of Column 1810 having an inside filled with Beads 1808 having PPase immobilized thereon and Beads 1809 having apyrase immobilized thereon. Even if an untreated reagent is prepared depending on the measuring conditions, its solution is treated while being passed through Column 1810 and impurities PPi and ATP are automatically removed prior to measurement. FIG. 17D illustrates one example of Removal part 1805 in the form of Column 1812 wherein PPase or apyrase has been directly immobilized on Porous material 1811 grown directly inside of the column. The column 1812 is filled with beads having a diameter φ of about 3 μm made of polystyrene, fused silica, glass or the like. The whole volume of the column is set at about 5 to 10 mL. The acting time is adjusted by selecting the flow rate in accordance with a solution to be treated. For example, the total amount of PPase in the column, for example, having a capacity of 10 mL is set at about 30 mU (U: unit of activity, unit), a flow rate of a reagent solution set at about 0.5 mL/min is sufficient for degradation and removal of an impurity PPi. Removal part 1805 which is equipped with Filter holder 1807 permitting release or exchange of the column therethrough is convenient in either case.

Concerning feeding of a reagent solution, direct feeding (pumping) by using a syringe or piezoelectric device or indirect feeding via a compressed gas may be adopted. The solution may be sucked while setting the pressure on the side of Dispenser 1803 at negative.

In this Embodiment, (1) DNA sequence analysis of a plurality of nucleic acid templates which are different each other, (2) analysis of single nucleotide polymorphisms at a predetermined site of a plurality of nucleic acid templates which are different each other, and (3) simultaneous analysis of a plurality of single nucleotide polymorphisms of one nucleic acid template. In (1), to reaction vessels having nucleic acid templates therein, respectively, a primer, reagents necessary for complementary strand extension reaction and reagents necessary for luminescence reaction are fed simultaneously, followed by successive addition of four nucleic acid substrates (dNTP:N=A,T,G,C) or analogues thereof. In (2), a mixed solution of four nucleic acid substrates is fed simultaneously from Reagent dispenser 1803. In (3), it is possible to add a plurality of primers successively to reaction vessels having therein the same nucleic acid template or to successively add a plurality of primers to reaction vessels having, therein, different nucleic acid templates, respectively. In any case, DNA sequence is analyzed by detecting, by Photosensor 1804, chemiluminescence from each reaction vessel as a result of complementary strand extension.

A feature of a method of analysis of DNA sequence is as follows:

(a) A method of analysis of DNA sequence, which comprises a first step of adding pyrophosphatase to a solution containing deoxyadenosine 5′-triphosphosphate, deoxythymidine 5′-triphosphate, deoxyguanosine 5′-triphosphate and deoxycytidine 5′-triphosphate, thereby degrading pyrophosphoric acid contained in the solution,

a second step of removing or inactivating the pyrophosphatase in the solution, and

a step of detecting, by chemiluminescence-reaction using a DNA primer, DNA polymerase and the solution obtained in the second step, pyrophosphoric acid generated by the extension reaction of the DNA primer hybridized to the target nucleic acid via a complementary strand.

(b) A method of analysis of DNA sequence as described in (a), wherein any one of deoxyadenosine 5′-triphosphate, deoxythymidine 5′-triphosphate, deoxyguanosine 5′-triphosphate and deoxycytidine 5′-triphosphate is replaced by an analogue thereof.

(c) A method of analysis of DNA sequence as described in (a), wherein the second or third base from the 3′terminus of the DNA primer has been replaced by a base not complementary to a base sequence of a predetermined region of the target nucleic acid.

(d) A method of analysis of DNA sequence as described in (a), wherein the extension reaction is conducted by degrading the strand extended by the extension reaction at the 5′ terminus by 5′ → 3′ exonuclease reaction and repeating complementary strand hybridization of the primer to the target nucleic acid.

(e) A method of analysis of DNA sequence, which comprises:

a first step of adding pyrophosphatase to a solution containing deoxyadenosine 5′-triphosphate, deoxythymidine 5′-triphosphate, deoxyguanosine 5′-triphosphate and deoxycytidine 5′-triphosphate, thereby degrading the pyrophosphoric acid contained in the solution,

a second step of removing or inactivating the pyrophosphatase in the solution, and

hybridizing, through a complementary strand, a first oligomer—which has complementary strain extending capacity and falls within a range of five bases to eight bases—and a second oligomer—which is hybridized, through a complementary strand, to the target nucleic acid and has no complementary strand extending capacity—in series to the target nucleic acid, carrying out extension reaction of the first oligomer by using DNA polymerase and the solution obtained in the second step, and detecting the pyrophosphoric acid formed by the extension reaction by chemiluminescence-reaction.

(f) A method of analysis of DNA sequence as described in (e), wherein any one of deoxyadenosine 5′-triphosphate, deoxythymidine 5′-triphosphate, deoxyguanosine 5′-triphosphate and deoxycytidine 5′-triphosphate is replaced with an analogue thereof.

(g) A method of analysis of DNA sequence as described in (e), the second or third base from the 3′terminus of the first oligomer has been replaced with a base not complementary to the base sequence of a predetermined region of the target nucleic acid.

A feature of a reagent kit is as follows:

(a) A reagent kit comprising pyrophosphatase in each of one or more solutions containing different deoxynucleotides, respectively, or each of one or more solutions containing different deoxynucleotides, respectively, at least one of which is an analogue thereof.

(b) A reagent kit as described in (a), further comprising DNA polymerase.

(c) A reagent kit comprising deoxyadenosine 5′-α-thio-triphosphosphate or deoxyadenosine 5′-α-thio-triphosphate, deoxythymidine 5′-triphosphate, deoxyguanosine 5′-triphosphate, deoxycytridine 5′-triphosphate, and pyrophosphatase.

(d) A reagent kit as described in (c), wherein the pyrophosphatase has been immobilized onto a solid.

(e) A reagent kit, which comprises at least one of DNA polymerase, DNA primer, adenosine 5′-phosphosulfate, ATP sulfurylase, luminescent enzyme, luminescent substrate and apyrase, and pyrophosphatase.

(f) A reagent kit as described in (e), wherein the pyrophosphatase and/or apyrase has been immobilized onto a solid.

A feature of an apparatus for analysis of DNA sequence is as follows:

A DNA sequence analyzer, which comprises a reaction chip equipped with a plurality of different sections on its solid surface for holding a target nucleic acid and a DNA primer to be hybridized thereto; means for feeding each of the plurality of sections with a reagent for causing complementary strand extension reaction with the primer as a starting point; and a photo detector for detecting chemiluminescence emitted making use of the reagent and pyrophosphoric acid generated by the complementary strand extension reaction which starts with the primer hybridized to the target DNA, wherein the reagent is fed through a removal part for removing an impurity pyrophosphoric acid or adenosine 5′-triphosphate from the reagent.

The method of analysis of DNA sequence, reagent kit and DNA sequence analyzer according to the present invention enables detection of SNPs of template DNA at high sensitivity in a short time. In addition, by the pretreatment or use of the reagent kit of the present invention, impurities contained in reagents used for the method of analysis of DNA sequence of the present invention or impurities such as inorganic pyrophosphoric acid (PPi) generated by the thermal degradation of a nucleic acid substrate can be degraded and removed, which makes it possible to carry out analysis of an existing ratio of SNPs with high reliability and high precision. Furthermore, the analysis method of DNA sequence of the present invention enables measurement of a large number of templates in a short time and materializes a DNA detection system with a high velocity and high throughput.

1. A method of analysis of DNA sequence, which comprises degrading, by pyrophosphatase, pyrophosphoric acid contained in a reagent used for extension reaction of a DNA primer hybridized to a target nucleic acid through a complementary strand and/or degrading, by apyrase, adenosine 5′-triphosphate contained in the reagent;
conducting the extension reaction, and
detecting pyrophosphoric acid generated by the extension reaction.

2. A method of analysis of DNA sequence according to claim 1, wherein the pyrophosphatase and/or the apyrase has been immobilized on a solid.

3. A method of analysis of DNA sequence, which comprises adding pyrophosphatase to one or more solutions which contain different deoxynucleotides, respectively, or to one or more solutions which contain different deoxynucleotides, respectively, at least one of which is an analogue thereof, thereby degrading pyrophosphoric acid contained in the solutions, and
extending a DNA primer, which has been hybridized to a target nucleic acid via a complementary strand, by using the DNA primer, DNA polymerase and at least one of the solutions obtained in said step and detecting pyrophosphoric acid thus generated by the extension reaction by chemiluminescence-reaction.

4. A method of analysis of DNA sequence, which comprises adding pyrophosphatase to one or more solutions which contain different deoxynucleotides, respectively, or to one or more solutions which contain different deoxynucleotides, respectively, at least one of which is an analogue thereof, thereby degrading pyrophosphoric acid contained in the solutions, and
extending a DNA primer, which has been hybridized to a target nucleic acid via a complementary strand, by using the DNA primer, DNA polymerase and at least one of the solutions obtained in said step, converting pyrophosphoric acid thus generated by the extension reaction into adenosine 5′-triphosphate in the presence of adenosine 5′-phosphosulfate and ATP sulfurylase, and detecting luminescence caused by chemiluminescence-reaction containing the adenosine 5′-triphosphate, a luminescence-enzyme and a luminescence substrate.

5. A method of analysis of DNA sequence according to claim 4, further comprising, after the first step, a step of removing or inactivating the pyrophosphatase in each of the solutions.

6. A method of analysis of DNA sequence according to claim 4, wherein the first step comprises adding the pyrophosphatase to at least one of the DNA-primer-containing solution, the DNA-polymerase-containing solution, the luminescence-enzyme-containing solution, the luminescence-substrate-containing solution, the adenosine 5′-phosphosulfate-containing solution and the ATP-sulfurylase-containing solution, thereby degrading the pyrophosphoric acid contained in at least one of said solutions, and/or adding apyrase to degrade adenosine 5′-triphosphate contained in at least one of said solutions.

7. A method of analysis of DNA sequence according to claim 6, further comprising removing or inactivating the pyrophosphatase and/or apyrase contained in the pyrophosphatase- and/or apyrase-added solution.

8. A method of analysis of DNA sequence according to claim 7, wherein the pyrophosphatase and/or apyrase has been immobilized on a solid.

9. A method of analysis of DNA sequence according to claim 4, wherein the base at the 3′terminus of the primer is complementary to the base one base behind the 3′terminus site of single nucleotide polymorphism of the target nucleic acid.

10. A method of analysis of DNA sequence according to claim 4, wherein the second or third base from the 3′terminus of the DNA primer has been substituted with a base not complementary to the base sequence of the target nucleic acid.

11. A method of analysis of DNA sequence, which comprises:
a first step of adding pyrophosphatase to each of a solution containing deoxyadenosine 5′-α-thiotriphosphate, a solution containing deoxythymidine 5′-triphosphate, a solution containing deoxyguanosine 5′-triphosphate and a solution containing deoxycytidine 5′-triphosphate, thereby degrading pyrophosphoric acid contained in each of the solutions;
a second step of removing or inactivating the pyrophosphatase in each of the solutions, and
a third step of extending a DNA primer, which has been hybridized to a target nucleic acid via a complementary strand, by using the DNA primer, DNA polymerase and at least one of the solutions obtained in said second step, converting pyrophosphoric acid thus generated by the extension reaction into adenosine 5′-triphosphate in the presence of adenosine 5′-phosphosulfate and ATP sulfurylase, and detecting luminescence caused by chemiluminescence-reaction containing the adenosine 5′-triphosphate, luciferase and luciferin.

12. A method of analysis of DNA sequence, which comprises:
a first step of adding pyrophosphatase to a solution containing deoxyadenosine 5′-α-thiotriphosphate, deoxythymidine 5′-triphosphate, deoxyguanosine 5′-triphosphate and deoxycytidine 5′-triphosphate, thereby degrading the pyrophosphoric acid contained in the solution;
a second step of removing or inactivating the pyrophosphatase in each of the solutions, and
a third step of extending a DNA primer, which has been hybridized to a target nucleic acid via a complementary strand, by using the DNA primer, DNA polymerase and at least one of the solutions obtained in said second step, converting pyrophosphoric acid thus generated by the extension reaction into adenosine 5′-triphosphate in the presence of adenosine 5′-phosphosulfate and ATP sulfurylase, and detecting luminescence caused by chemiluminescence-reaction containing the adenosine 5′-triphosphate, luciferase and luciferin.

13. A method of analysis of DNA sequence according to claim 12, wherein the second or third base from the 3′terminus of the DNA primer has been substituted by a base not complementary to the base sequence of the target nucleic acid.

14. A method of analysis of DNA sequence according to claim 12, wherein the extension reaction is conducted by degrading the strand, which has been extended by the extension reaction, from the 5′terminus thereof by the 5′ → 3′ exonuclease reaction and repeating complementary strand hybridization of the DNA primer to the target nucleic acid.
